(19) United States
(12) Patent Application Publication
(10) Pub. No.: US 2014/0024119 A1
(43) Pub. Date: Jan. 23, 2014

(54) CELL REPROGRAMMING COMPOSITION COMPRISING REX1 AND AN INDUCED PLURIPOTENT STEM CELL PRODUCTION METHOD USING THE SAME

(71) Applicants: Yee Sook Cho, Yuseong-gu (KR); Mi Young Son, Yuseong-gu (KR)

(72) Inventors: Yee Sook Cho, Yuseong-gu (KR); Mi Young Son, Yuseong-gu (KR)

(73) Assignee: KOREA RESEARCH INSTITUTE OF BIOSCIENCE AND BIOTECHNOLOGY, Yuseong-gu, Daejeon (KR)

(21) Appl. No.: 13/979,674

(22) PCT Filed: Jan. 16, 2012
(86) PCT No.: PCT/KR12/00363

§ 371 Date: Jul. 15, 2013

(30) Foreign Application Priority Data
Jan. 14, 2011 (KR) 10-2011-0004247

(51) Int. Cl. C12N 5/074

(52) U.S. Cl. CPC C12N 5/0696
USPC 435/455; 435/377

(57) ABSTRACT

A reprogramming inducing composition including the Rex1 protein or a nucleic acid molecule coding for the Rex1 protein for producing induced pluripotent stem cells from body cells or non-embryonic cells through a reprogramming process. A method for producing induced pluripotent stem cells by using the Rex1 is also disclosed.

### BACKGROUND OF THE INVENTION

1. Field of the Invention

The present invention relates to a reprogramming-inducing composition comprising the Rex1 protein or a nucleic acid molecule encoding the Rex1 protein for producing induced pluripotent stem cells from somatic cells or non-embryonic cells through a reprogramming process, and a method for producing induced pluripotent stem cells by using Rex1.

2. Description of the Related Art

In 2006, professor Yamanaka's team at Kyoto University in Japan was the first to develop a reprogramming technology capable of producing induced pluripotent stem cells from somatic cells through the combined overexpression of reprogramming transcription factors (Oct4, Sox2, Klf4, and cMyc) that play a pivotal role in pluripotency (Cell, 126: 663-676, 2006). Many countries in the world fiercely compete to lead the development of cell therapy products and new drugs based on this technology. The reprogramming technology allows the production of induced pluripotent stem cells having the characteristics of human embryonic stem cells from the autologous-somatic cells that are acquired in a relatively easy way, thereby causing less bodily injury and discomfort to a patient, leading to a remarkable evolution in methods for establishing a personalized, autologous pluripotent stem cell line from a patient's somatic cells. The reprogramming technology is also recognized as the best solution for addressing bioethical issues and immune compatibility problems that can be caused by the use of human embryonic stem cells, thereby providing infinite possibilities for its future application to regenerative medical fields.

In spite of such advantages, current reprogramming technology still has the problems of risk of tumor formation, low reprogramming efficiency, and a long time frame needed for the reprogramming process, which should be solved for the practical application to the development of cell therapy products and new drugs. Normally, production of induced pluripotent stem cells by overexpressing Yamanaka factors, namely, a set of genes Oct4, Sox2, cMyc and Klf4 in human somatic cells using a retroviral expression system, is recognized as yielding the highest reprogramming efficiency, but the reprogramming efficiency was also as low as 0.01% to 0.1% in this case. Because c-Myc and Klf4, which are known as important factors for improving reprogramming efficiency in the current reprogramming technology, have oncogenic function, there are safety concerns about the formation of tumors when they are used in the production of induced pluripotent stem cells and the differentiated cells therefrom are then used in the development of cell therapy products. Accordingly, to meet the quality and quantity requirements of practical applications, there is a need to find a new reprogramming factor as an alternative to the known oncogenic factors capable of remarkably improving the reprogramming efficiency and to subsequently develop a technology capable of practically utilizing the factor in the reprogramming process.

On the other hand, Rex1 (reduced expression protein 1, ZFP42) transcription factor is a member of the zinc finger protein family, and was first found by observation of its specific reduced expression in the induced differentiation of pluripotent murine F9 embryonal carcinoma (EC) cells (Mol Cell Biol, 9: 5623-5659, 1989). However, there has been no report of a relationship between Rex1 and the reprogramming of differentiated cells.

### Technical Problem

The present inventors have made many efforts to develop a novel reprogramming factor capable of solving the problems of the previous reprogramming technology and a reprogramming system using the same. As a result, they found that reprogramming efficiency can be effectively improved and that the time and the number of reprogramming factors required for reprogramming can be remarkably reduced by using the Rex1 transcription factor for the reprogramming process of producing induced pluripotent stem cells from human somatic cells, and that the induced pluripotent stem cells produced by use of Rex1 retain the pluripotent characteristics of human embryonic stem cells, thereby completing the present invention. They also found that Rex1 can be used in a variety of differentiated cells without lineage-specific restriction, because Rex1 shows similar reprogramming-improving effects in various somatic cells derived from different lineages. Additionally, the present inventors found that such reprogramming-improving effects of the Rex1 transcription factor are attributed to improvements in cell proliferation inhibition and cell-cycle arrest which occur as reprogramming barriers in the previous reprogramming technology, thereby completing the present invention.

### SUMMARY OF THE INVENTION

An object of the present invention is to provide a composition for inducing reprogramming differentiated cells into induced pluripotent stem cells, comprising the Rex1 protein or a nucleic acid molecule encoding the Rex1 protein.

Another object of the present invention is to provide use of the composition for reprogramming differentiated cells into induced pluripotent stem cells.

Still another object of the present invention is to provide a method for producing the reprogrammed induced pluripotent stem cells by introducing the reprogramming-inducing factor comprising the Rex1 protein or a nucleic acid molecule encoding the Rex1 protein into the differentiated cells.

### BRIEF DESCRIPTION OF THE DRAWINGS

FIG. 1 is the results of Real-time RT-PCR (FIGS. 1a-d) and immunostaining (FIG. 1e) showing expression patterns of lineage-specific markers (FIGS. 1a and b) and pluripotency-specific markers (FIGS. 1c and d) to characterize the undifferentiated and differentiated states of stem cells. In this experiment, human embryonic stem cells (hES), human induced pluripotent stem cells (hiPS) and human embryonal carcinoma cells (hEC) were tested as the undifferentiated stem cells, and human foreskin fibroblasts (hFF), embryonic bodies (early EB) formed by suspension culture for 5 days, embryonic bodies (late EB) formed by suspension culture for 28 days, hES (RA-hES) formed by retinoic acid-induced differentiation, hES-neural progenitor cells (hES-NP), cardiomyocytes (hES-CM), osteocytes (hES-OS), and endothelial cells (hES-EC) were tested as the differentiated cells. Further, expression of partially reprogrammed hiPS (partial hiPS) was analyzed for comparison.

FIG. 2 is the result of agarose gel electrophoresis of Rex1 gene product (A), and a map of a retroviral expression vector pMXs cloned with Rex1 (B).

FIG. 3 is a diagram showing a summary of the reprogramming technology used in the present invention, in which images at the bottom represent somatic cells (CRL2097) used in the reprogramming process, established hiPS, and ALP-positive hiPS cells.

FIG. 4 is the result of ALP staining showing the changes in reprogramming efficiency when four reprogramming factors including Oct4, Sox2, c-Myc and Klf4 were used together with Rex1 factor in reprogramming process. The value on the graph represents mean±S.E. The somatic cell used in this test was hFF. Low; retrovirus MOI=1, High; retrovirus MOI=5.

FIG. 5 shows contribution of Rex1 factor to reprogramming process of mouse embryonic fibroblasts. The left panel represents ALP staining images of the cell groups that underwent the reprogramming process. The right panel represents the number of ALP-positive colonies, and the value represents mean±S.E.

FIG. 6 shows contribution of Rex1 factor to reprogramming process of human foreskin fibroblasts in combinations with the previous reprogramming factors, in which the minimum and optimum combinations of the reprogramming factors required for reprogramming were examined. The upper panel shows the result of ALP-staining of the cells that underwent the reprogramming process. The lower panel represents the number of ALP-positive colonies, and the value on the graph represents mean±S.E.

FIG. 7 shows the contribution of Rex1 factor to the reprogramming process of human foreskin fibroblasts, which was confirmed through the transfection amount of the reprogramming factors. Changes in the reprogramming efficiency according to the infection amount (MOI) of the Rex1 gene were examined by the hES-like morphology and the number of ALP-positive colonies, and shown in the graph. The value on the graph represents mean±S.E. FIG. 7c is a representative image showing ALP-positive colonies after reprogramming of human foreskin fibroblasts using each of the marked reprogramming factors. O; Oct4, S; Sox2, M; cMyc, K; Klf4, R; Rex1.

FIG. 8 is an image and a graph showing the results of ALP staining to examine the contribution of Rex1 factor to the reprogramming process, in which reprogramming was induced under the conditions of specifically suppressing Rex1 expression using Rex1-specific shRNA, and the value on the graph represents mean±S.E. *p<0.05.

FIG. 9 shows the characteristics of hiPS (pOSM-hiPS) partially reprogrammed using three reprogramming factors, Oct4, Sox2, and c-Myc. At this time, the somatic cell used in the reprogramming was hFF. FIG. 9a shows cell morphology, ALP staining, and immunostaining for the expression patterns of pluripotency markers in pOSM-hiPS (size bar=200 μm). FIG. 9b is the results of Real-time RT-PCR showing the expression patterns of the pluripotency markers in hFFs, H9 hES, and pOSM-hiPS (piPS). FIG. 9c is the result of analyzing DNA methylation status in the promoter regions of Oct4 and Rex1 in pOSM-hiPS. The empty circle and solid circle represent demethylated CpG and methylated CpG, respectively, and the ratio of methylated CpG was expressed as %.

FIG. 10 shows cell morphology, ALP staining, and immunostaining for the expression patterns of pluripotency markers in hiPS (OSKM-hiPS) fully reprogrammed by four reprogramming factors Oct4, Sox2, cMyc and Klf4, and hiPS (OSMR-hiPS) fully reprogrammed by Oct4, Sox2, cMyc and Rex1 (size bar=200 μm). At this time, somatic cell used was hFF.

FIG. 11 is the result of Real-time RT-PCR showing the expression patterns of the pluripotency markers in OSKM-hiPS and OSMR-hiPS. Real-Time RT-PCR was performed using specific PCR primers prepared for comparison analysis of total (Total), endogenous (Endo) and retroviral exogenous transgene (Trans) expressions of the corresponding genes.

FIG. 12 is the result of RT-PCR showing genomic integration of exogenous reprogramming factors in OSKM-hiPS and OSMR-hiPS.

FIG. 13 is the result of karyotype analysis of the cultured OSMR-iPSC.

FIG. 14 is the result of analyzing promoter methylation of Oct4 and Rex1 transcription factors in H9 hES, hFF, OSKM-hiPS (4F-hiPS), and OSMR-hiPS (3F+R-hiPS). The empty circle and solid circle represent demethylated CpG and methylated CpG, respectively, and the ratio of methylated CpG was expressed as %.

FIG. 15 is the images showing the result of immunostaining, which was performed to examine differentiation capacity of OSKM-hiPS and OSMR-hiPS into 3 germ layers (ectoderm, endoderm, and mesoderm) in vitro using lineage-specific marker antibodies.

FIG. 16 is the result of Real-time RT-PCR to examine differentiation capacity of OSKM-hiPS and OSMR-hiPS into 3 germ layers in vitro using lineage-specific markers.

FIG. 17 is the images showing teratoma formation to examine differentiation capacity of OSMR-hiPS into 3 germ layers in vivo.

FIG. 18 shows contribution of Rex1 factor to reprogramming of human neural progenitor cells (FIG. 18a) and human mesenchymal stem cells (FIG. 18b) into induced pluripotent stem cells. Changes in the reprogramming efficiency by combinations of the previous reprogramming factors (O, S, K, and/or M) and Rex1 factor were examined by ALP-staining analysis, and shown in the graph. The value on the graph represents mean±S.E. *p<0.05, **p<0.01.

FIG. 19 shows embryonic stem cell-like morphology, ALP staining, and expression patterns of the pluripotency markers in the induced pluripotent stem cells (1F+R-hiPS) prepared from human neural progenitor cells using two factors Oct4 and Rex1, and in the induced pluripotent stem cells (2F+R-hiPS) prepared from human mesenchymal stem cells using three factors Oct4, Sox2, and Rex1.

FIG. 20 shows methylation patterns of Oct4, Nanog, and Rex1 promoter regions in the induced pluripotent stem cells (1F+R-hiPS) prepared from human neural progenitor cells using two factors Oct4 and Rex1, and in the induced pluripotent stem cells (2F+R-hiPS) prepared from human mesenchymal stem cells using three factors Oct4, Sox2, and Rex1. Empty circle and black circle represent demethylated CpG and methylated CpG, respectively, and the ratio of methylated CpG was expressed as %.

FIG. 21 is the results of Real-time RT-PCR (A) and Western blotting (B) showing specific suppression of Rex1 expression by Rex1-specific shRNA in the NCCIT human embryonal carcinoma cell line at the gene and protein levels, respectively. Protein bands detected in the Western blot were quantified and the mean values thereof are shown in the graph. *p<0.05.

FIG. 22 is the result of Real-time RT-PCR showing gene expression patterns of pluripotency markers and lineage-specific differentiation markers in the Rex1 expression-suppressed human embryonal carcinoma cell line.

FIG. 23 is the result of microarray analysis showing expression patterns of pluripotency markers and lineage-specific differentiation markers in the Rex1 expression-suppressed human embryonal carcinoma cell line.

FIG. 24 is the result of analyzing the microarray data based on “Ingenuity Pathway Analysis software”, and is the result of analyzing biological function (FIG. 24a) and canonical pathway (FIG. 24b) of the genes that show at least 2-fold difference in the expression levels in Rex1 expression-suppressed cell lines (shREX1-10, shREX1-11, and shREX1-13), compared to the cell line treated with non-target control shRNA (shNT) as a negative control group.

FIG. 25 is the result of microarray showing gene expression profiles of cell cycle G2/M progression-related genes specific to the Rex1 expression-suppressed human embryonal carcinoma cell line.

FIG. 26 shows cell growth inhibition in the Rex1 expression-suppressed cell lines (shREX1-10, shREX1-11, and shREX1-13) (** P<0.01, *P<0.05). Changes in the cell growth were examined by periodically determining the number of cells during cell culture (day 1, 3, 5) (*p<0.05, **p<0.01).

FIG. 27 shows changes in cell cycle distribution in the Rex1 expression-suppressed cell lines (shREX1-10, shREX1-11, and shREX1-13). Upper panel shows the result of analyzing cell cycle distribution, and lower panel is a graph showing % of cells distributed in G1, S, and G2/M.

FIG. 28 is the results of p-histone H3 fluorescent immunostaining (A) and Western blotting (B) showing G2 phase arrest of the Rex1 expression-suppressed cell lines (shREX1-10, shREX1-11, and shREX1-13) during G2/M phase.

FIG. 29 is the result of Western blotting showing the expression patterns of G2/M progression-related proteins in the Rex1 expression-suppressed cell lines (shREX1-10, shREX1-11, and shREX1-13).

FIG. 30 is the results of Real-time RT-PCR (upper) and Western blotting (lower) showing increased expressions of cyclin B1 and cyclin B2 by Rex1 overexpression at the gene and protein levels, respectively. Protein bands detected in the Western blot were quantified and the mean values thereof are shown in the graph. * P<0.01.

FIG. 31 is the result of Real-time RT-PCR showing increased expressions of cyclin B1 and cyclin B2 at the gene level by use of Rex1 in reprogramming, compared to no use of Rex1 as a control group. Oct4+Sox2+cMyc=3F, Oct4+Sox2+cMyc+Klf4=4F, Oct4+Sox2+cMyc+Rex1=3F+R, Oct4+Sox2+cMyc+Klf4+Rex1=4F+R; *** P<0.01, ** P<0.05, * P<0.1.

### DETAILED DESCRIPTION OF THE PREFERRED EMBODIMENTS

In one aspect, the present invention provides a composition for inducing reprogramming differentiated cells into induced pluripotent stem cells, comprising the Rex1 (reduced expression protein 1) protein or a nucleic acid molecule encoding the Rex1 protein.

In another aspect, the present invention provides a use of the composition for reprogramming differentiated cells into induced pluripotent stem cells.

As used herein, the term “Rex1 protein”, also known as ZFP42, is a member of the zinc finger protein family and is a transcription factor that was first found in murine F9 embryonal carcinoma cells.

Rex1 used in the composition of the present invention may be any Rex1 derived from a human or animal such as mouse, and is preferably, human Rex1. Further, the Rex1 protein of the present invention may be a protein having an amino acid sequence of the wild-type thereof and a variant thereof. The variant of the Rex1 protein means a protein which has a different amino acid sequence from that of the native Rex1 due to deletion, insertion, non-conservative or conservative substitution of one or more amino acid residues, or combinations thereof. The variant may be a functional equivalent having biological activity identical to a native protein, or, if desired, may be made by altering the physicochemical property of the native form. For example, the variant may have increased structural stability or increased physiological activity against physical and chemical environments.

As used herein, the term “nucleic acid molecule encoding the Rex1 protein” refers to a nucleotide sequence encoding the Rex1 protein of the wild-type or the above described variant type thereof, and may be modified by substitution, deletion, or insertion of one or more bases, or combinations thereof. It may be isolated from the natural source or prepared by a chemical synthetic method.

The Rex1 transcription factor of the present invention has an effect of improving reprogramming efficiency of differentiated cells into induced pluripotent stem cells, and is a reprogramming inducer that can be used for reprogramming as an alternative to prior reprogramming factors such as an oncogene, Klf4 or the like. Such effect of Rex1 was first discovered by the present inventors.

According to a preferred embodiment, the nucleic acid molecule encoding the Rex1 protein may be included in an expression vector.

According to a preferred embodiment, the Rex1 protein may be a protein that is expressed from a cell line in vitro by using the expression vector including the nucleic acid molecule encoding the Rex1 protein. The expression vector may be a Baculovirus expression vector, a Mammalian expression vector or a Bacterial expression vector, and the cell line may be an insect cell line, a mammalian cell line, or a bacterial cell line. However, the expression vector and the cell line usable in the present invention are not limited thereto.

As used herein, the term “expression vector” refers to a genetic construct that contains essential regulatory elements to which a gene insert is operably linked such that a desired gene is expressed in a suitable host cell. The expression vector of the present invention may be used for the purpose of introducing the reprogramming-inducing factor into the differentiated cells.

The expression vector of the present invention may include a signal sequence or a leader sequence for targeting membranes or secretion as well as expression regulatory elements, such as a promoter, an operator, an initiation codon, a stop codon, a polyadenylation signal and an enhancer, and can be constructed in various forms depending on the purpose thereof. The promoter of the vector may be constitutive or inducible. In addition, the expression vector includes a selectable marker that allows the selection of host cells containing the vector, and a replicable expression vector includes a replication origin. The expression vector may be self-replicable, or may be integrated into the host DNA. The vector may include a plasmid vector, a cosmid vector, an episomal vector, a viral vector or the like, and preferably a viral vector. Examples of the viral vector may include those derived from retrovirus such as HIV (Human immunodeficiency virus) MLV (Murineleukemia virus) ASLV (Avian sarcoma/leukosis), SNV (Spleen necrosis virus), RSV (Rous sarcoma virus), MMTV (Mouse mammary tumor virus) etc., adenovirus, adeno-associated virus, herpes simplex virus, sendai virus or the like, but are not limited thereto.

According to a preferred embodiment, the nucleic acid molecule encoding the Rex1 protein may be messenger RNA (mRNA).

As used herein, the term “reprogramming” or “de-differentiation” means a process capable of restoring or converting differentiated cells in a different state such as cells with no or little differentiation capacity into cells with a new type of differentiation capacity. Such cell reprogramming mechanism involves the removal of epigenetic (DNA state associated with changes in gene function that occur without a change in the nucleotide sequence) marks in the nucleus, followed by establishment of a different set of marks, and different cells and tissues acquire different gene expression programs during the differentiation and growth of multicellular organisms. With respect to the objects of the present invention, the ‘reprogramming’ may encompass all processes of reversing differentiated cells with a differentiation capacity of 0% to less than 100% into undifferentiated cells, and preferably, a process of restoring or converting differentiated cells with a differentiation capacity of 0% or partially differentiated cells with a differentiation capacity of more than 0% to less than 100% into cells with a differentiation capacity of 100%.

As used herein, the term “reprogramming-inducing factor” is a substance that induces reprogramming of partially or fully differentiated cells into induced pluripotent stem cells having a potential to differentiate into a new type of cells, and includes the Rex1 protein or the nucleic acid molecule encoding the Rex1 protein. The reprogramming-inducing factor may include any substance without limitation, as long as it is able to induce reprogramming of differentiated cells. The reprogramming factor may be selected depending on the type of cells to be fully differentiated.

According to a preferred embodiment, the composition of the present invention may further include one or more proteins selected from the group consisting of Oct4, Sox2, KlF4, c-Myc, Nanog and Lin-28, or one or more nucleic acid molecules encoding these proteins as the reprogramming-inducing factor, but is not limited thereto.

According to a preferred embodiment, the differentiated cells may be those derived from various animals such as human, monkey, pig, horse, cow, sheep, dog, cat, mouse, rabbit or the like, and preferably, those derived from human. However, the differentiated cells that can be reprogrammed into the induced pluripotent stem cells are not limited thereto.

According to a preferred embodiment, the differentiated cells may be somatic cells or somatic stem cells.

As used herein, the term ‘somatic cell’ refers to a cell that constitutes an adult body, and has restricted differentiation capacity and self-renewal capacity. According to a preferred embodiment, the somatic cells may be those that constitute the human skin, hair, or fat, and preferably, human fibroblasts, but are not limited thereto.

According to a preferred embodiment, the somatic stem cells may be hematopoietic stem cells, mammary stem cells, intestinal stem cells, mesenchymal stem cells, endothelial stem cells, neural stem cells, olfactory adult stem cells, or neural crest stem cells, and preferably, neural stem cells (neural progenitor cells) or mesenchymal stem cells, but are not limited thereto.

As used herein, the term “induced pluripotent stem cells” or “reprogrammed stem cells” refer to cells that are prepared by establishing undifferentiated stem cells having pluripotency similar to that of embryonic stem cells from differentiated cells using a reprogramming technology. Induced pluripotent stem cells have characteristics almost similar to those of embryonic stem cells. In detail, induced pluripotent stem cells are similar to embryonic stem cells in morphology, gene and protein expression patterns, pluripotency in vivo and in vitro, teratoma formation, formation of chimeric mice after blastocyst injection, and/or germ-line transmission of genes.

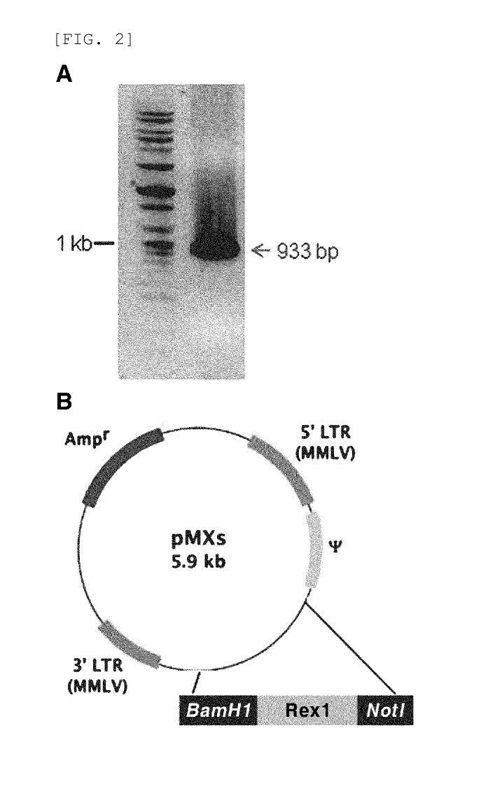

In one embodiment of the present invention, in order to examine contribution of Rex1 to reprogramming of the human fibroblasts having no differentiation capacity into induced pluripotent stem cells, the previous reprogramming factors including Oct4, Sox2, Klf4, cMyc or the like were overexpressed together with Rex1 overexpression, and the reprogramming efficiency was examined by analysis of cell morphology and ALP-positive colonies. The results showed that the reprogramming efficiency was increased depending on the concentration of exogenous Rex1, which was confirmed by the increased number of ALP-positive colonies (for Rex1, 4.9-fold increase at an MOI of 1 and 2.6-fold increase at an MOI of 5). In particular, when the reprogramming was induced with Rex1, colonies of induced pluripotent stem cells were formed 5 days early (see Examples 11-3 and FIG. 4).

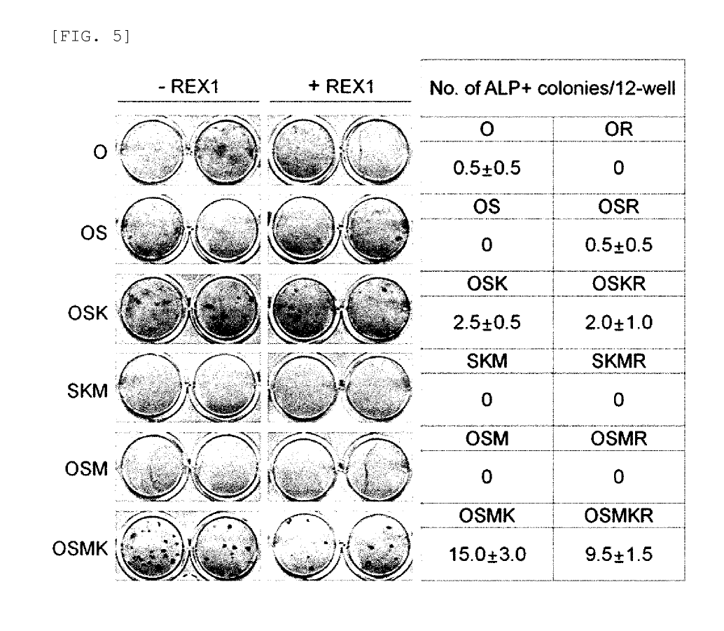

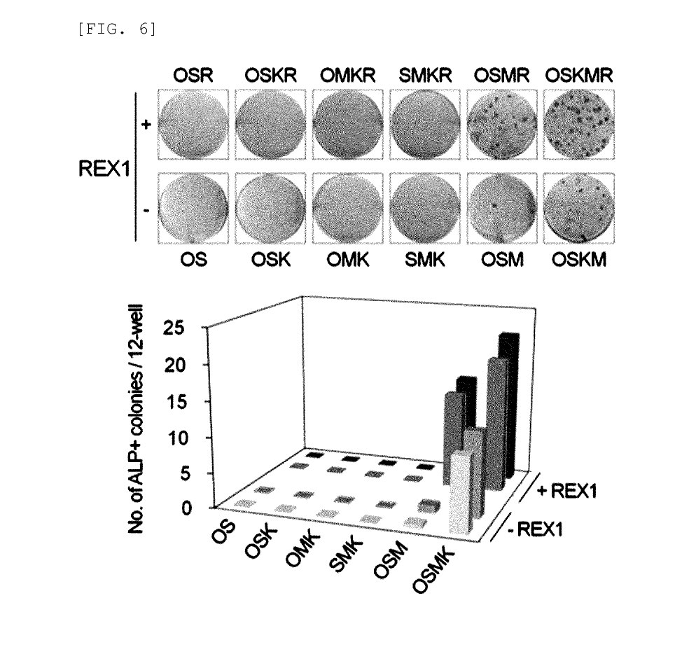

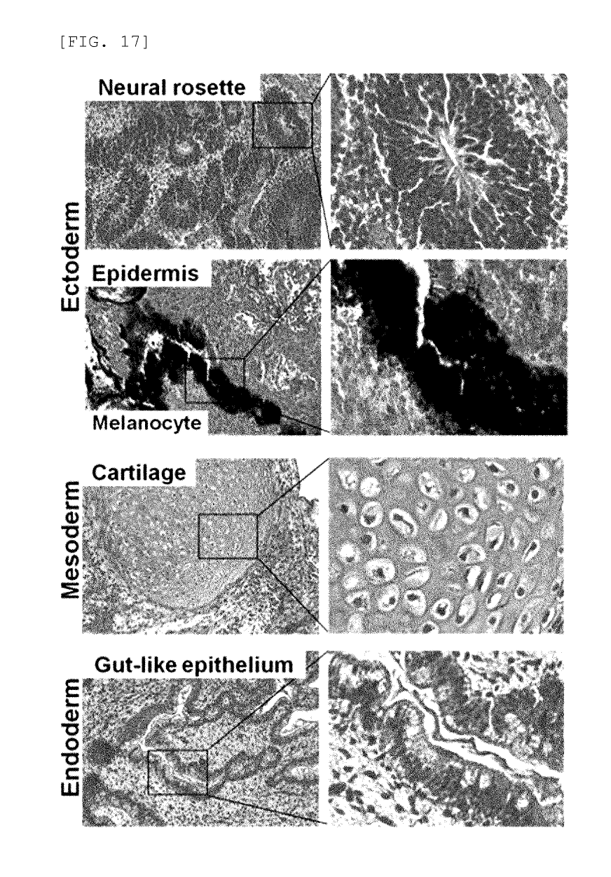

Further, the possibility of using Rex1 as an alternative to the reprogramming factor was examined, and the result showed that although the reprogramming of human fibroblasts having no differentiation capacity was induced with Rex1 in the absence of Klf4, Rex1 showed higher reprogramming efficiency than Klf4 (see Examples 12 and 13 and FIGS. 6 and 7), indicating that Rex1 can be used as the alternative to the reprogramming-inducing factor Klf4, and shows remarkably higher reprogramming efficiency than Klf4, thereby improving the conventional reprogramming technology. Furthermore, in one embodiment of the present invention, it was confirmed that Rex1 contributes to effective reprogramming of neural progenitor cells (FIG. 18a) and mesenchymal stem cells (FIG. 18b) possessing restricted differentiation capacity into induced pluripotent stem cells in a state of pluripotency, and in particular, Rex1 improves the reprogramming efficiency and shortens the time required for reprogramming (Example 17 and FIGS. 18 to 20). Although it is impossible to induce reprogramming of human neural progenitor cells using a single factor Oct4, their reprogramming was effectively induced 7 days early by use of two factors Rex1 and Oct4 (0.039% of reprogramming efficiency). Full reprogramming of human mesenchymal stem cells was effectively induced 10-14 days early by use of three factors Oct4, Sox2, and Rex1 (0.039% of reprogramming efficiency). These results indicate that Rex1 can be used as the reprogramming factor to induce effective reprogramming of somatic stem cells and multipotent stem cells as well as somatic cells into induced pluripotent stem cells.

In another aspect, the present invention provides a method for producing induced pluripotent stem cells reprogrammed from differentiated cells. In detail, the preparation method of the present invention includes the steps of (a) introducing the reprogramming-inducing factor including the Rex1 protein or the nucleic acid molecule encoding the Rex1 protein into the differentiated cells, and (b) culturing the cells of step (a).

According to a preferred embodiment, the method of the present invention may further include the step of (c) isolating embryonic stem cell-like colonies from the culture broth obtained in step (b).

As the method for introducing the reprogramming-inducing factor into cells in step (a), any method typically used in the art for providing cells with nucleic acid molecule or protein can be used without limitation, and preferably, a method for adding the reprogramming-inducing factor to the culture broth of differentiated cells or a method for directly injecting the reprogramming-inducing factor into differentiated cells may be used. In this regard, the reprogramming-inducing factor to be used may be in a form of a virus obtained from a packaging cell transfected with a viral vector having the corresponding gene, a messenger RNA produced by in vitro transcription, or a protein produced in various cell lines.

The method for directly injecting the reprogramming-inducing factor into differentiated cells may be any method known in the art, and may be properly selected from microinjection, electroporation, particle bombardment, direct intramuscular injection, insulator and transposon techniques, but is not limited thereto.

The viral vector may be a vector derived from retrovirus, lentivirus, adenovirus, adeno-associated virus, herpes simplex virus, sendai virus or the like, but is not limited to, preferably, a retroviral vector, and more preferably, a retroviral vector pMXs. Further, the packaging cell may be selected from various cells known in the art, depending on the used viral vector, but is not limited to, preferably a GP2-293 packaging cell.

To produce the reprogramming-inducing factor protein, the nucleic acid molecule encoding the protein can be expressed in vitro using a baculovirus expression vector system in an insect cell line, a mammalian expression vector system in a mammalian cell line or a bacterial expression vector system in a bacterial cell line.

The reprogramming-inducing factor may be selected depending on the cell type to be differentiated. According to one preferred embodiment, the reprogramming-inducing factor may further comprise one or more proteins selected from the group consisting of Oct4, Sox2, KlF4, c-Myc, Nanog and Lin-28, or one or more nucleic acid molecules encoding these proteins.

The differentiated cells are the same as those described in the reprogramming-inducing composition.

The culture of the cells may be performed according to an appropriate medium and culture conditions known in the art. The culture procedure may be easily adjusted depending on the selected cells by those skilled in the art.

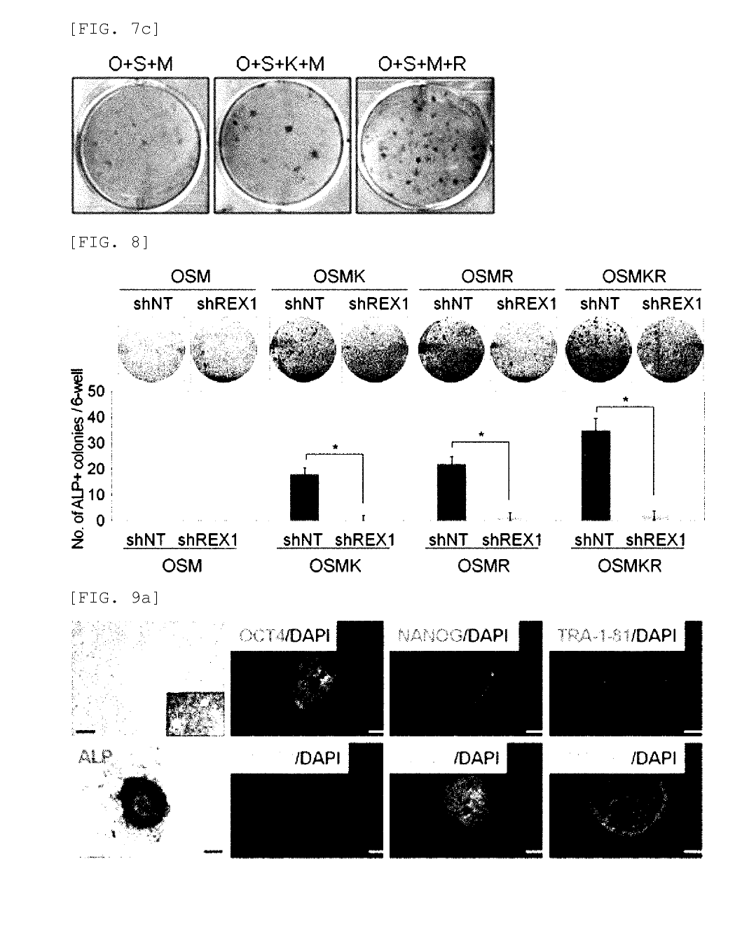

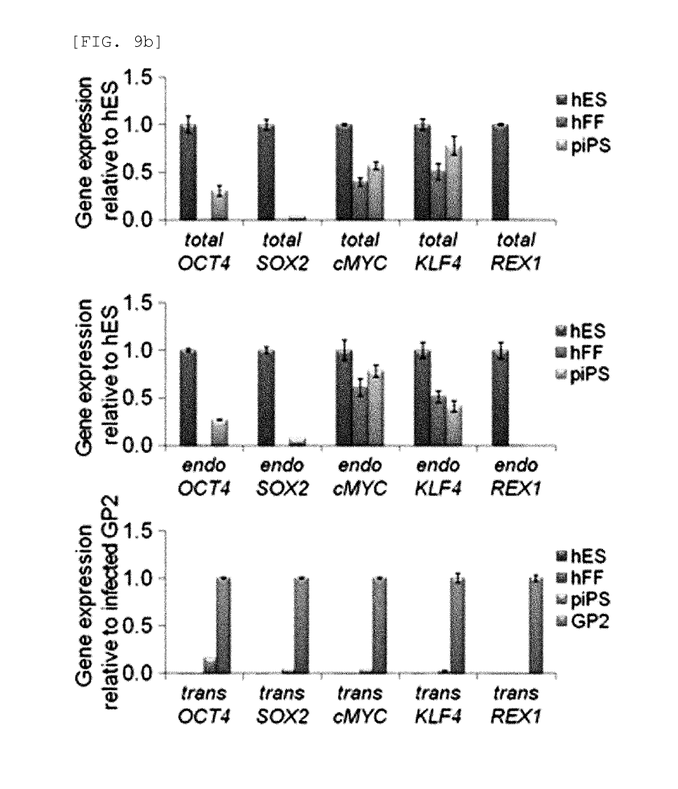

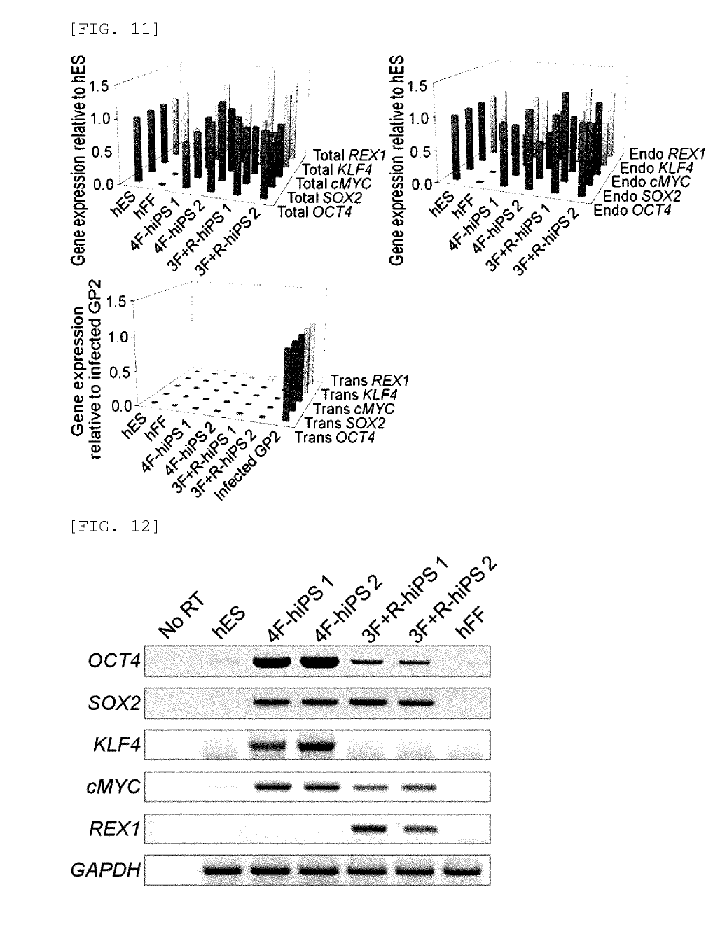

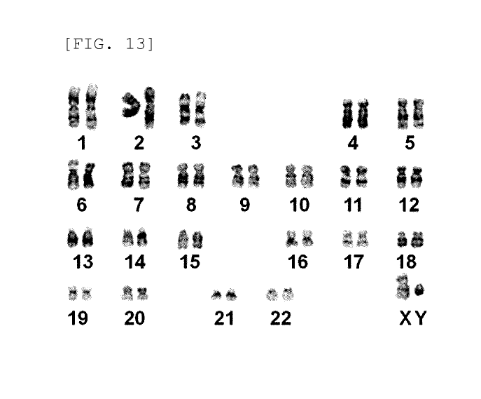

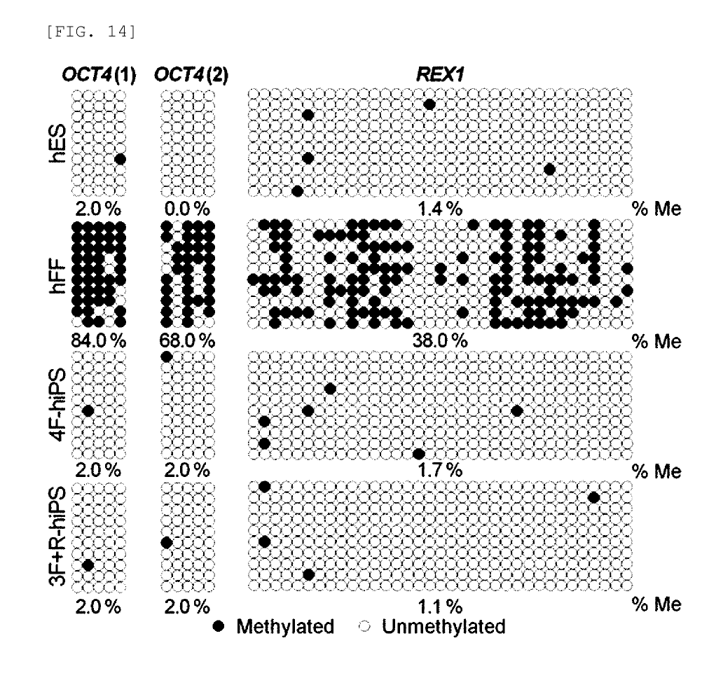

In one embodiment of the present invention, Rex1 was used as the reprogramming-inducing factor to prepare Oct4, Sox2, cMyc and Rex1-induced pluripotent stem cell lines, and characterization of the stem cells was performed by ALP staining and immunostaining. The result showed that the induced pluripotent stem cell lines were very similar to or otherwise indistinguishable from each other in the morphology or the pluripotency marker expression patterns (see FIGS. 10-11). RT-PCR was performed to examine the genomic integration of the exogenous Oct4, Sox2, c-Myc, Rex1 in reprogrammed cells. Further, in the methylation profile of promoter regions of the pluripotency marker genes, the induced pluripotent stem cell line showed similar patterns to human embryonic stem cell line (see FIG. 14). The differentiation potential of embryoid body derived from the induced pluripotent stem cell line was examined. The results showed that the induced pluripotent stem cells established by use of Rex1 had a potential to differentiate into all 3 germ layers in vivo and in vitro (see Example 16 and FIGS. 15-17), and maintained a normal karyotype even after continuous long-term culture (FIG. 13).

Hereinafter, the preferred Examples are provided for better understanding of the present invention. However, these Examples are for illustrative purposes only, and the invention is not intended to be limited by these Examples.

### Example 1

### Culture of Human Embryonic Stem Cells

Human embryonic stem cells (hESC) H9 (NIH Code, WA09; WiCell Research Institute, Madison, Wis.) and induced pluripotent stem cells (hiPSC) were cultured on γ-radiated mouse embryonic fibroblasts using hESC culture medium composed of 80% DMEM/F12, 20% knockout serum alternative (Invitrogen, Carlsbad, Calif.), 1% non-essential amino acids (Invitrogen), 1 mM L-glutamine (Invitrogen), 0.1 mM β-mercaptoethanol (Sigma, St. Louis, Mo.) and 6 ng/ml basic fibroblast growth factor (Invitrogen). The cells were sub-cultured every 5 to 6 days using 1 mg/ml collagenase IV (Invitrogen). Human newborn foreskin fibroblasts (hFF, ATCC, catalog number CRL-2097; American Type Culture Collection, Manassas, Va.) were cultured in DMEM containing 10% FBS (Invitrogen), 1% non-essential amino acids, 1 mM L-glutamine and 0.1 mM β-mercaptoethanol. Human neural progenitor cells (hNPs, ReNcell CX Immortalized cells, Millipore, SCC007) were cultured in Complete ReNcell NSC medium supplemented with FGF-2 (20 ng/ml) and EGF (20 ng/ml). Mouse embryonic fibroblasts were cultured in DMEM containing 10% FBS (Invitrogen), 1% non-essential amino acids and 0.1 mM β-mercaptoethanol.

### Example 2

### RNA Extraction, Reverse Transcription and PCR Analysis

Total RNAs were isolated from the produced cells using an RNeasy Mini kit (Qiagen, Valencia, Calif.), and then reverse transcription was performed using a SuperScript First-strand synthesis system kit (Invitrogen) according to the manufacturer's directions. Thereafter, semi-quantitative RT-PCR was performed using a platinum Tag SuperMix kit (Invitrogen) under the following conditions: at 94° C. for 3 min, 25 to 30 cycles of at 94° C. for 30 sec, at 60° C. for 30 sec and at 72° C. for 30 sec, elongation at 72° C. for 10 min. The primer sequences used are given in the following Table 1.

TABLE 1

| Gene | Primer (Forward) | Primer (Reverse) | SEQ ID NO. |
|---|---|---|---|
| Total | GAGAAGGATGTG | CAGAGGAAAGGAC | 10/11 |
| OCT4 | GTCCGAGTGTG | ACTGGTCCC | |
| Total | AGAACCCCAAGA | ATGTAGGTCTGCG | 12/13 |
| SOX2 | TGCACAAC | AGCTGGT | |
| Total | ACCCTGGGTCTT | ACGATCGTCTTCC | 14/15 |
| KLF4 | GAGGAAGT | CCTCTTT | |
| Total | CCTACCCTCTCA | CTCTGACCTTTTG | 16/17 |
| cMYC | ACGACAGC | CCAGGAG | |
| Total | AATGCGTCATAA | TCAATGCCAGGTA | 18/19 |
| REX1 | GGGGTGAG | TTCCTCC | |
| Endo | GACAGGGGGAGG | CTTCCCTCCAACC | 20/21 |
| OCT4 | GGAGGAGCTAGG | AGTTGCCCCAAAC | |
| Endo | GGGAAATGGGAG | TTGCGTGAGTGTG | 22/23 |
| SOX2 | GGGTGCAAAAGA | GATGGGATTGGT | |
| | GG | G | |
| Endo | AGCCTAAATGAT | TTGAAAACTTTGG | 24/25 |
| KLF4 | GGTGCTTGGT | CTTCCTTGTT | |
| Endo | CGGGCGGGCACT | GGAGAGTCGCGTC | 26/27 |
| cMYC | TTG | CTTGCT | |
| Endo | TCAGTGACAAAC | CCTTCCTGTTGGA | 28/29 |
| REX1 | ATGCCTCA | AGCACAC | |

For transgene and genomic integration

| | | | |
|---|---|---|---|
| Trans | GAGAAGGATGTG | CCCTTTTTCTGGA | 30/31 |
| OCT4 | GTCCGAGTGTG | GACTAAATAAA | |
| Trans | GGCACCCCTGGC | TTATCGTCGACCA | 32/33 |
| SOX2 | ATGGCTCTTGGC | CTGTGCTGCTG | |
| | TC | | |
| Trans | ACGATCGTGGCC | TTATCGTCGACCA | 34/35 |
| KLF4 | CCGGAAAAGGAC | CTGTGCTGCTG | |
| | C | | |
| Trans | CAACAACCGAAA | TTATCGTCGACCA | 36/37 |
| cMYC | ATGCACCAGCCC | CTGTGCTGCTG | |
| | CAG | | |
| Trans | ACGTTTCGTGTG | TAACCTGAAAGCC | 38/39 |
| REX1 | TCCCTTTC | CACATCC | |

hESC markers

| | | | |
|---|---|---|---|
| NANOG | CAAAGGCAAACA | ATTGTTCCAGGTC | 40/41 |
| | ACCCACTT | TGGTTGC | |
| DNMT3B | ATAAGTCGAAGG | GGCAACATCTGAA | 42/43 |
| | TGCGTCGT | GCCATTT | |

Ectoderm lineage markers

| | | | |
|---|---|---|---|
| NCAM | AGGAGACAGAAA | GGTGTTGGAAATG | 44/45 |
| | CGAAGCCA | CTCTGGT | |
| SOX1 | GGGAAAACGGGC | CCATCTGGGCTTC | 46/47 |
| | AAAATAAT | AAGTGTT | |
| OTX1 | AGACGCATCAGA | CCAGACCTGGACT | 48/49 |
| | CCCTGAAGGACT | CTAGACTC | |
| TUBB3 | GGCCAAGTTCTG | CGAGTCGCCCACG | 50/51 |
| | GGAAGTCA | TAGTTG | |

Mesoderm lineage markers

| | | | |
|---|---|---|---|
| MSX1 | TCCTCAAGCTGC | TACTGCTTCTGGC | 52/53 |
| | CAGAAGAT | GGAACTT | |
| IGF2 | CAGACCCCCAAA | GCCAAGAAGGTGA | 54/55 |
| | TTATCGTG | GAAGCAC | |
| COL1A1 | GGACACAATGGA | TAACCACTGCTCC | 56/57 |
| | TTGCAAGG | ACTCTGG | |
| FOXF1 | AAAGGAGCCACG | AGGCTGAAGCGAA | 58/59 |
| | AAGCAAGC | GGAAGAGG | |
| HAND1 | TCCCTTTTCCGC | CATCGCCTACCTG | 60/61 |
| | TTGCTCTC | ATGGACG | |

Endoderm lineage markers

| | | | |
|---|---|---|---|
| HGF | gcatcaaatgtcagccct | caacgctgacatggaattc | 62/63 |
| | gg | c | |
| GATA6 | CCATGACTCCAA | ACGGAGGACGTGA | 64/65 |
| | CTTCCACC | CTTCGGC | |
| AMYLASE | GCTGGGCTCAGT | GACGACAATCTCT | 66/67 |
| | ATTCCCCAAAT | GACCTGAGTAG | |
| AFP | AGCAGCTTGGTG | CCTGACCTTGGCA | 68/69 |
| | GTGGATGA | CAGATCCT | |
| SOX17 | TTCGTGTGCAAG | GTCGGACACCACCG | 70/71 |
| | CCTGAGATG | AGGAA | |
| Cyclin B1 | TGGCGCTCCGAG | GTTGCAGCAGGGG | 72/73 |
| | TCACCAGG | CCGTAGG | |
| Cyclin B2 | AGGCGAGCATCA | GGGAGGCAAGGTC | 74/75 |
| | AAAGCCGGG | TTTGACGGC | |
| GAPDH | GAAGGTGAAGGT | GAAGATGGTGATG | 76/77 |
| | CGGAGTC | GGATTTC | |

### Example 3

### Specific Expression of Rex1 in Undifferentiated Pluripotent Stem Cells (Embryonic Stem Cells and Induced Pluripotent Stem Cell)

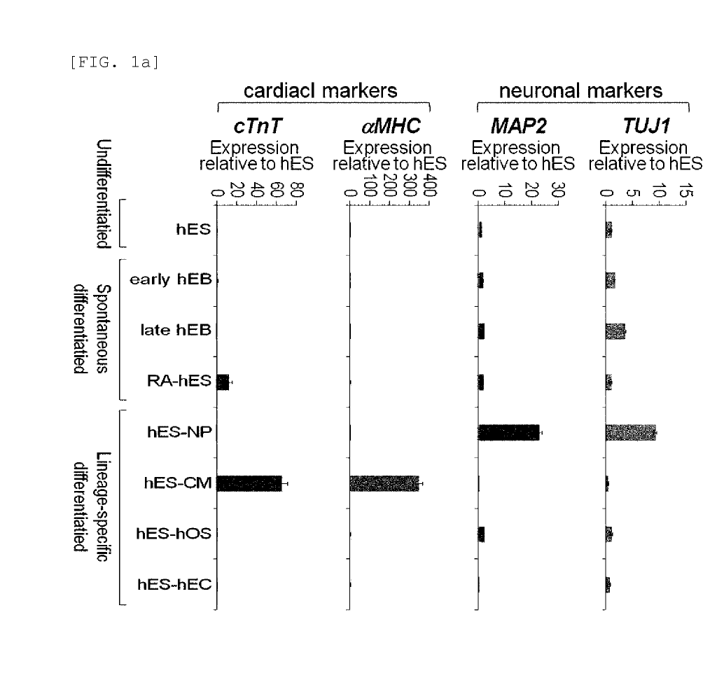

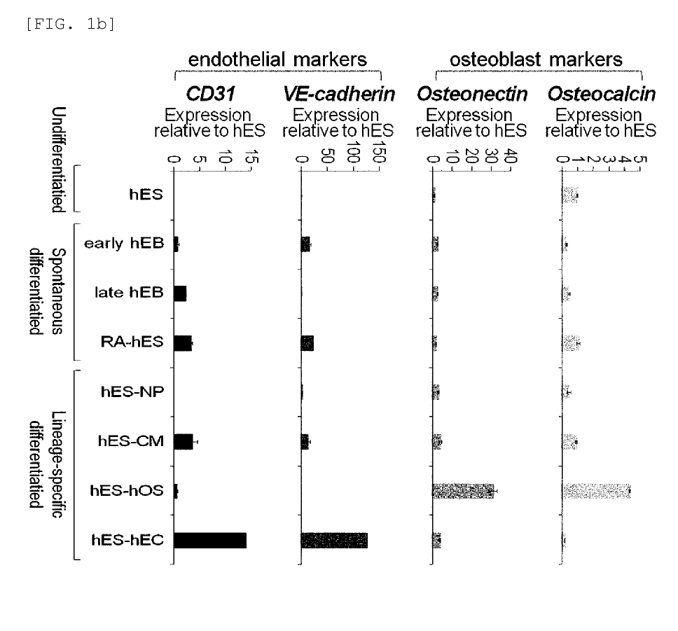

Rex1 mRNA expression patterns were examined in undifferentiated human embryonic stem cells and embryonic bodies spontaneously differentiated therefrom (early embryonic body differentiated for 5 days, and late embryonic body differentiated for 28 days), retinoic acid (RA)-treated human embryonic stem cells, and specific lineage cells derived from human embryonic stem cells. Characterization of specific lineage cells differentiated from human embryonic stem cells was confirmed by an increase in the lineage-specific marker expression (FIGS. 1a and 1b). Expressions of Rex1 and various pluripotency markers were examined by Real-time RT-PCR, and the results showed that Rex1 expression was markedly decreased or not observed in all differentiated cells, its expression was very low in the early differentiation stage, compared to other markers, and no expression was observed in partially reprogrammed human induced pluripotent stem cells (FIG. 1d). Rex1 protein expression was examined in undifferentiated and differentiated human pluripotent stem cells (hES, hiPS) and partially reprogrammed human induced pluripotent stem cells (phiPS) by immunostaining. The results showed that Rex1 protein was exclusively expressed in undifferentiated hES and fully reprogrammed undifferentiated hiPS, and not expressed in partially reprogrammed cells (phiPS) (FIG. 1e). These results suggest that Rex1 is a strictly acting undifferentiation-specific pluripotency marker that is able to detect early differentiation of human embryonic stem cells and induced pluripotent stem cells with high sensitivity.

### Example 4

### Construction of Human Rex1 Gene Expression Vector

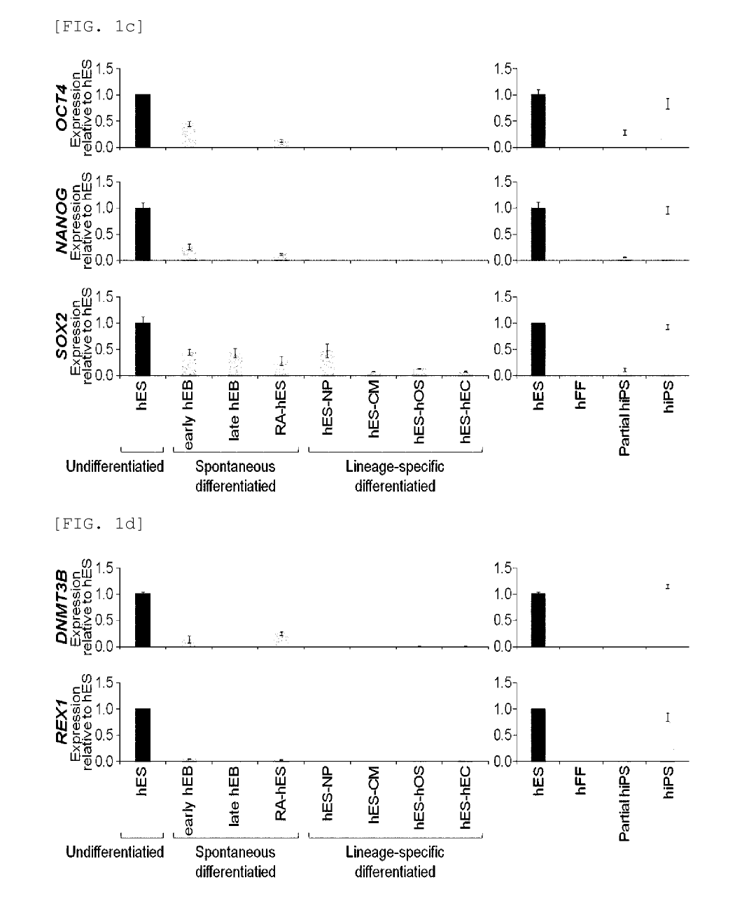

In order to acquire the Rex1 gene having the base sequence of SEQ ID NO. 1, cDNA prepared from RNA of human embryonic stem cells and a primer set of SEQ ID NOs. 2 and 3 were used to perform PCR, and PCR conditions are the same as in Example 2. Rex1 gene of 933 by in size was obtained by PCR (A of FIG. 2), and inserted into a pCR2.1-TOPO vector (Invitrogen) by a TA cloning method, and the Rex1-inserted pCR2.1-TOPO vector and a retroviral vector, pMXs vector were cleaved by treatment with restriction enzymes, respectively and ligated to prepare a gene expression vector pMXs-Rex1 for reprogramming (B of FIG. 2).

### Example 5

### Retrovirus Production and hiPSC Induction

pMXs vectors containing human cDNAs of Oct4 (POU5F1), Sox2, c-Myc (MYC) and KlF4, as described in the paper of Takahashi, K. et al. (Cell 131, 2007, 861-872), were purchased from Addgene. The Rex1-expressing retroviral vector was the same one as in Example 4. A packaging cell line GP2-293 was transfected with the retroviral vector DNA and VSV-G envelope vector using lipofectamine 2000. 24 hours after transfection, the medium was collected as the first virus-containing supernatant and replaced with a new medium, which was collected after 24 hours as the second virus-containing supernatant. The supernatants were sterilized through a 0.45 μm pore-size filter, and centrifuged at 20,000 rpm for 90 minutes, and stored at −70° C. until use. For generation of induced pluripotent stem cells, human foreskin fibroblasts (hFFs), human neural progenitor cells (hNPs), mouse embryonic fibroblasts (MEF) were seeded in a gelatin-coated E-well plate at a density of 1×10⁵ cells per well 6 hours before transduction, and infected with virus in the presence of polybrene (6 μg/ml). 5 days after transduction, hFFs or hNPCs were harvested by trypsinization, and re-plated onto the previously gelatin-coated 6-well plate having MEF as a feeder layer at a density of 5 to 6×10⁴ per well. For experiment under feeder-free conditions, the cells were seeded onto the Matrigel-coated 6-well plate at a density of 5 to 6×10⁴ per well. Next day, the medium for hFFs or hNPCs was replaced with hESC medium supplemented with 10 ng/ml of bFGF, and the medium for MEF cells was replaced with mESC medium [containing DMEM; Gibco-BRL, Gaithersburg, Md.; 15% fetal bovine serum, 100 μM non-essential amino acids (Invitrogen, Carlsbad, Calif.), 1 mM sodium pyruvate (Invitrogen), 100 μM 2-mercaptoethanol (Invitrogen), 1× antibiotic-antimycotic (Invitrogen)] supplemented with 1000 U/ml of leukemia inhibitory factor (ESGRO; Chemicon, Temecula, Calif.). The media were changed every other day. 20 days after transduction, hESC colonies were obtained, and transferred onto MEF feeder cells in a 12-well plate, and continuous proliferation culture was carried out by the hESC culture method of Example 1.

### Example 6

### Embryoid Body Differentiation

To examine differentiation potential of hESCs, human embryonic bodies (hEBs) were cultured in suspension using hEB medium (DMEM/F12 containing 10% serum alternative) in non-tissue culture treated Petri dishes. 5 days after suspension culture, embryonic bodies were transferred onto the gelatin-coated plate, and cultured using the hEB medium. Under the conditions as above, adherent cells on the bottom of the plate were left for further 15 days for differentiation, if necessary, by changing the medium.

### Example 7

### Immunocytochemistry

For immunostaining, cells were seeded on a Matrigel-coated 4-well Lab-Tek chamber slide (Nunc, Naperville, Ill.), and cultured for 5 days under the disclosed conditions. The cells were fixed in 4% paraformaldehyde for 15 minutes at room temperature (RT), and then washed with PBS/0.2% BSA, and permeabilized in PBS/0.2% BSA/0.1% Triton X-100 for 15 minutes, and then reacted with 4% normal donkey serum (Molecular Probes, Eugene, Oreg., USA) in PBS/0.2% BSA at room temperature for 1 hour. The cells were reacted with primary antibody diluted with PBS/0.2% BSA at 4° C. for 2 hours. After washing, the cells were reacted with FITC or Alexa594-conjugated secondary antibody (Invitrogen) in PBS/0.2% BSA at room temperature for 1 hour. The cells were counter-stained using 10 μg/ml of DAPI. The chamber slides were analyzed using an Olympus microscope or Axiovert 200M microscope (Carl Zeiss, Gottingen, Germany). The antibodies used are given in the following Table 2.

TABLE 2

| Antibodies | Catalog No. | Company | Dilution |
|---|---|---|---|
| hESC marker | | | |
| anti-Oct4 | sc-9081 | Santa Cruz | 1:150 for |
| | | Biotechnology | immunostaining |
| anti-Nanog | AF1997 | R&D | 1:100 for |
| | | | immunostaining |
| anti-SSEA-3 | MAB1434 | R&D | 1:20 for |
| | | | immunostaining |
| anti-SSEA-4 | MAB1435 | R&D | 1:50 for |
| | | | immunostaining |
| anti-TRA1-60 | MAB4360 | Chemicon | 1:80 for |
| | | | immunostaining |
| anti-TRA1-81 | MAB4381 | Chemicon | 1:80 for |
| | | | immunostaining |
| In vitro | | | |
| differentiation | | | |
| anti-TUJ1 | PRB-435P | Covance | 1:500 for |
| | | | immunostaining |
| anti-NESTIN | MAB5326 | Chemicon | 1:100 for |
| | | | immunostaining |
| anti-FoxA2 | 07-633 | Chemicon | 1:100 for |
| | | | immunostaining |
| anti-SOX17 | MAB1924 | R&D | 1:50 for |
| | | | immunostaining |
| anti-α-SMA | A5228 | Sigma | 1:400 for |
| | | | immunostaining |
| anti-desmin | AB907 | Chemicon | 1:30 for |
| | | | immunostaining |

### Example 8

### Promoter Methylation Profiling of Reprogramming Transcription Factors

For characterization of human embryonic stem cells and induced pluripotent stem cells established by retroviral transfection, promoter methylation status of human embryonic stem cell-specific transcription factors, Oct3/4 and Nanog was analyzed. Genomic DNAs were extracted from human embryonic stem cells and induced pluripotent stem cells that had cultured in the hESC culture medium for 6 days using a DNA extraction kit (Qiagen Genomic DNA purification kit). Bisulfite sequencing was carried out in three steps. Step 1 is to modify DNA using sodium bisulfite, step 2 is to amplify the desired gene regions (normally, promoter regions) by PCR, and step 3 is to analyze DNA methylation by sequencing of PCR products. In the step of DNA modification using sodium bisulfite, a commercial kit, EZ DNA Methylation Kit (Zymo Research) was used. During treatment of DNA with bisulfite, unmethylated cytosine residues are converted to uracil, while methylated cytosine residues are unaffected. Therefore, unmethylated and methylated DNAs can be distinguished by PCR using cytosine- and uracil-specific primers. The primers used are given in the following Table 3.

TABLE 3

| | | | |
|---|---|---|---|
| | | | Accession |
| Gene | Primer (Forward) | Primer (Reverse) | No. |

For bisulfate sequencing

| | | | |
|---|---|---|---|
| bi | ATTTGTTTTTTG | CCAACTATCTTCAT | NM_002701 |
| Oct4- | GGTAGTTAAAGGT | CTTAATAACATCC | |
| 3) | | | |
| SEQ | 4 | 5 | |
| ID | | | |
| NO. | | | |
| bi | GGATGTTATTAAGA | CCTAAACTCCCCT | NM_002701 |
| Oct4 | TGAAGATAGTTGG | TCAAAATCTATT | |
| SEQ | 6 | 7 | |
| ID | | | |
| NO. | | | |
| bi | TGGTTAGGTTGGT | AACCCACCCTTAT | NM_024865 |
| Nanog | TTTAAATTTTTG | AAATTCTCAATTA | |
| SEQ | 8 | 9 | |
| ID | | | |
| NO. | | | |

A PCR reaction mixture containing 1 μg of bisulfite-treated DNA, 0.25 mM/1 of dNTP, 1.5 mM/1 of MgCl₂, 50 pM of primers, 1×PCR buffer, 2.5 units of Taq polymerase (Platinum Taq DNA polymerase, Invitrogen, Carlsbad, Calif., USA) was prepared in a final volume of 20 μl. PCR was carried out under the conditions of at 95° C. for 10 min, 40 cycles of at 95° C. for 1 min, at 60° C. for 1 min, at 72° C. for 1 min, and final reaction at 72° C. for 10 min. The PCR products were confirmed on a 1.5% agarose gel. After gel elution, the resultant was cloned into the pCR2.1-TOPO vector (Invitrogen). The methylated and unmethylated base sequences were analyzed by sequencing using M13 primers.

### Example 9

### Karyotype Analysis

The cultured human reprogrammed stem cells were confirmed by G-banding analysis. The representative images were captured by ChIPS-Karyo (Chromosome Image Processing System, GenDix).

### Example 10

### Culture System for Induced Pluripotent Stem Cell Induction

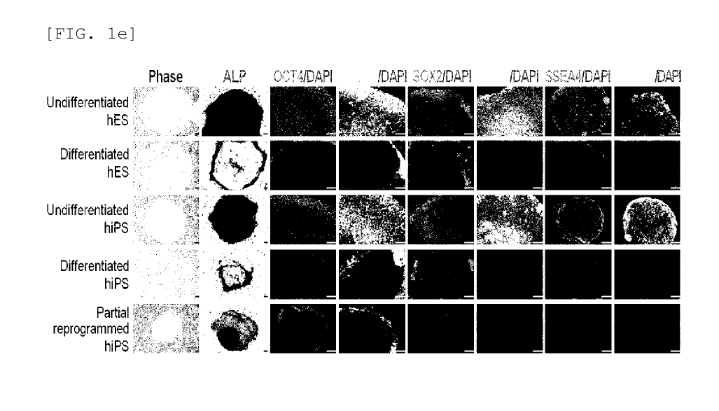

On the basis of Yamanaka' reprogramming system (Cell 126, 2006, 663-676), a reprogramming technique was established, and in order to examine transfection efficiency, a pMXs-EGFP-Rheb-IP vector was used. The number of GFP-positive cells was determined by FACS to calculate MOI (multiplicity of infection) of virus. Somatic cells were transfected with the virus concentrate at an MOI of 1 to 5. Next day after transfection, the medium was replaced. 5 days, cells were detached using Trypsin-EDTA, and transferred to feeder cells, followed by culture in a somatic cell culture medium. Next day, the medium was replaced with the hESC culture medium, and periodically replaced with a new culture medium for continuous culture. After approximately 2-4 weeks, generation of cells having the shape of human embryonic stem cells was observed, and an induced pluripotent stem cell line stably maintained was established by sub-culture (FIG. 3).

### Example 11

### Increase of Reprogramming Efficiency by Rex1

11-1. ALP Staining (Alkaline Phosphatase Staining)

ALP staining was performed using a commercial ALP kit according to the manufacturer (Sigma)'s instructions. The images of ALP-positive cells were recorded by HP Scanjet G4010. The bright field images were also obtained using an Olympus microscope (IX51, Olympus, Japan).

11-2. Reprogramming Efficiency Test

In order to measure the reprogramming efficiency into human induced pluripotent stem cells, the number of colonies with the human embryonic stem cell-like shape formed on the MEF feeder or the Matrigel-coated 6-well plate after reprogramming culture were determined, and divided by the number of cells initially inoculated. The number of colonies stained with the pluripotency marker ALP was determined, and divided by the number of cells initially inoculated, thereby calculating the reprogramming efficiency. At this time, the experiment was repeated three times.

11-3. Reprogramming Efficiency by Rex1

In order to examine changes in the efficiency of Rex1-mediated reprogramming of human fibroblast cells, changes in reprogramming efficiency by Rex1 addition were examined, compared to those by addition of Oct4, Sox2, Klf4 and cMyc-expressing retroviral vectors as standard. As a result, when Rex1 was used at an MOI of 1 and 5, 4.9-fold and 2.6-fold increases in the reprogramming efficiency was observed, respectively, which was confirmed by the increased number of ALP-positive colonies (FIG. 4). It was found that the Rex1-mediated reprogramming efficiency was increased in a Rex1 concentration-dependent manner. Further, when Rex1 was added in hFF, colonies of induced pluripotent stem cells were formed 5 days early.

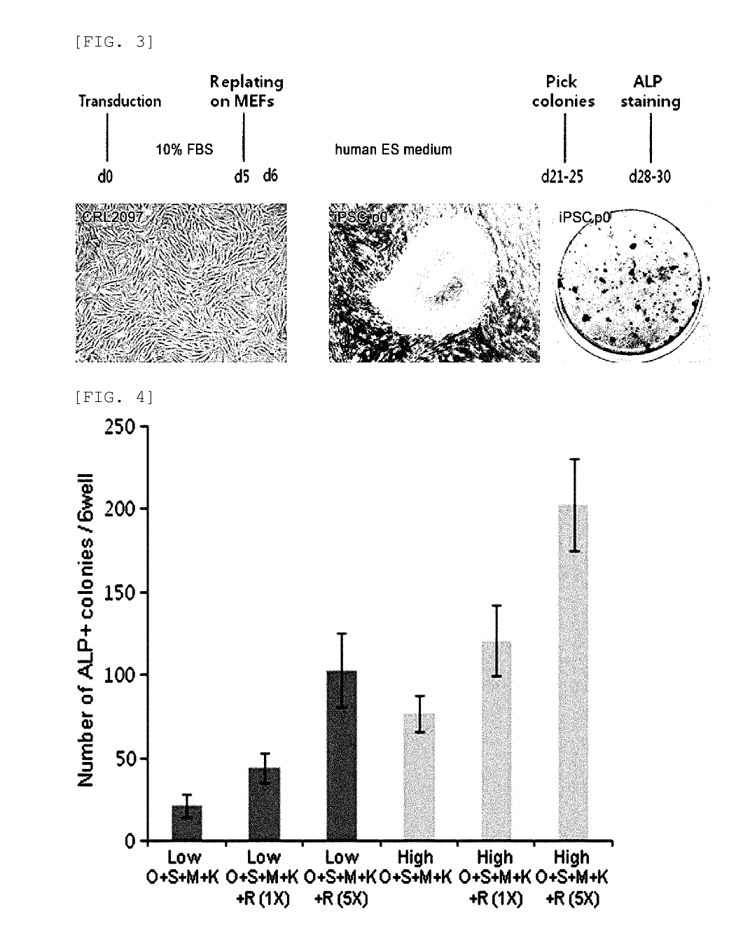

With respect to the Yamanaka' reprogramming system, mouse embryonic fibroblasts (MEFs) obtained from Fbx15^{βgeo/βgeo} were used to screen 24 candidate genes and the reprogramming factors Oct4, Sox2, c-Myc and Klf4 essential for reprogramming were selected therefrom (Cell 126, 2006, 663-676). However, Rex1 was excluded from the reprogramming factors during a subsequent selection procedure. The present inventors added different combinations of Oct4, Sox2, c-Myc and Klf4 to MEFs and examined changes in Rex1-mediated reprogramming. There was no specific change by the addition, in particular, no reprogramming-improving effect (FIG. 5). These results indicate differences in the reprogramming process between human and mouse, suggesting that Rex1 shows different functions in early development and pluripotency regulation of stem cells between human and mouse systems.

### Example 12

### Rex1 as Alternative to Klf4 Reprogramming Factor

It was examined whether the previous reprogramming factors can be replaced or reduced by Rex1. Although the orphan nuclear receptor Nr5a2 is reported to replace Oct4 in MEF cells, there was no report on human cells, and Oct4 is generally known as an irreplaceable essential factor in reprogramming (Cell Stem Cell. 2010 Feb. 5; 6(2):167-74). The reprogramming efficiency was measured in the same manner as in Examples 11-1 and 11-2. As shown in FIG. 6, a small number of ALP-positive colonies were observed when Oct4, Sox2 and cMyc were only used. However, an induced pluripotent stem cell line fully reprogrammed therefrom could not be established, and these colonies were found to be a partially reprogrammed cell line (pOSM-iPSC). However, when Klf4 or Rex1 was added thereto, fully reprogrammed induced pluripotent stem cell lines (OSMK-iPSCs, OSMR-iPSCs) were established. The number of ALP-positive colonies was compared between OSMK-iPSCs and OSMR-iPSCs. As a result, the use of Rex1 showed higher efficiency than the use of Klf4, and led to successful reprogramming under the use of a smaller amount of c-Myc (FIG. 6). Further, Rex1 fully replaced Klf4 and partially replaced c-Myc upon reprogramming of human fibroblasts, but could not replace Oct4 and Sox2. These results indicate that essential reprogramming factors for human fibroblasts are Oct4, Sox2, cMyc, Klf4, or Oct4, Sox2, cMyc, and Rex1 (FIGS. 6 and 7).

### Example 13

### Concentration-Dependent Reprogramming Effect of Reprogramming Factor Rex1

As Rex1 was confirmed to function as the reprogramming factor, it was examined whether the reprogramming-improving effect of Rex1 was increased by ectopic expression. To this end, cells were infected with viruses containing Oct4, Sox2 and c-Myc genes at a high MOI (5 MOI) and a low MOI (1-0.5 MOI), respectively and then changes in the reprogramming efficiency according to Rex1 concentration were examined. As a result, when the cells were infected with viruses containing Oct4, Sox2 and cMyc genes at a low MOI (1 MOI), Rex1 increased the reprogramming efficiency in a concentration-dependent manner, and the treatment of Rex1 at an MOI of 5 increased the efficiency approximately 19-fold and approximately 4.8-fold, compared to OSM-iPSCs and OSMK-iPSCs, respectively (FIG. 7). In particular, in order to further quantify these effects, experiment was carried out by addition of various concentrations of the reprogramming factors and Rex1. As shown in FIG. 7, the reprogramming efficiency was increased, as the concentrations of Oct4, Sox2, cMyc and Rex1 were all increased. However, when Oct4, Sox2 and cMyc were added at a ratio of 3:1:1, and Rex1 was added at an MOT of 5, the most synergistic reprogramming effect was observed. In addition, OSMR-iPSCs and OSMK-iPSCs were compared at the same MOT. As a result, the reprogramming factor increased the number of ALP-positive colonies 1.6-fold at an MOT of 1 and 1.8-fold at an MOT of 5 in OSMR-iPSCs, compared to OSMK-iPSCs. These results indicate that Rex1 can be used as the alternative to Klf4, and is also excellent in terms of reprogramming efficiency.

### Example 14

### Reduction of Reprogramming Efficiency by Rex1-Specific shRNA

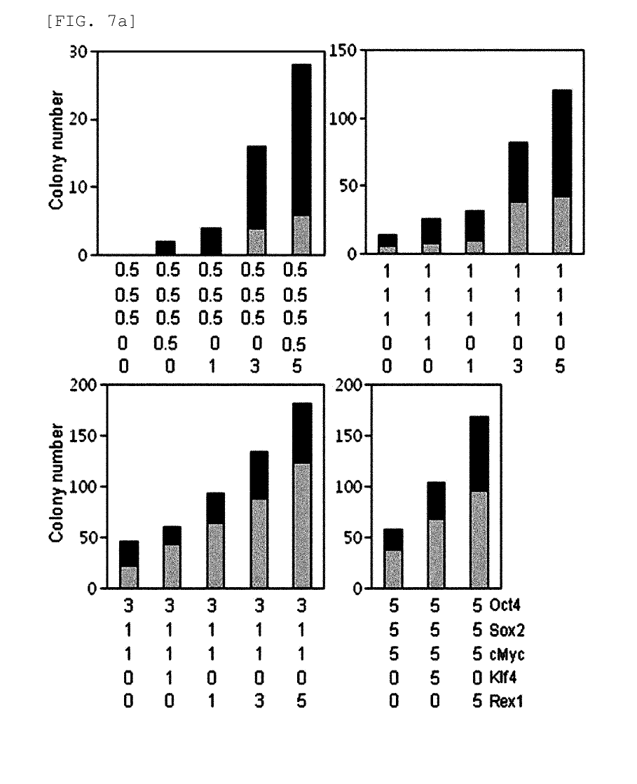

The reprogramming effect of Rex1 was also confirmed by specifically suppressing Rex1 expression using a short hairpin RNA (shRNA) delivery system by lentivirus. Rex1-specific lentivirus shRNA was prepared by the method of the following Example 18-2. The effects were investigated by co-transfection of human fibroblasts with various combinations of reprogramming factors, Oct4, Sox2, cMyc, Klf4 and Rex1, and Rex1-shRNA. As shown in FIG. 8, when ectopic expressions of OSM, OSMK, OSMR, and OSKMR were induced by retrovirus, no ALP-positive colonies were observed in OSM, and ALP-positive colonies were observed in OSMK or OSMR treated with Klf4 or Rex1, and the number of ALP-positive colonies was increased in OSKMR. However, even though ALP-positive colonies were observed, the number of ALP-positive colonies was significantly reduced in the group reprogrammed by co-treatment with Rex1-shRNA, compared to a negative control group treated with non-target shRNA (shNT) (p<0.01, FIG. 8). These results suggest that the reprogramming efficiency is reduced by suppressing endogenous expression and/or ectopic expression of Rex1.

### Example 15

### Characterization of Partially Reprogrammed Human Induced Pluripotent Stem Cells

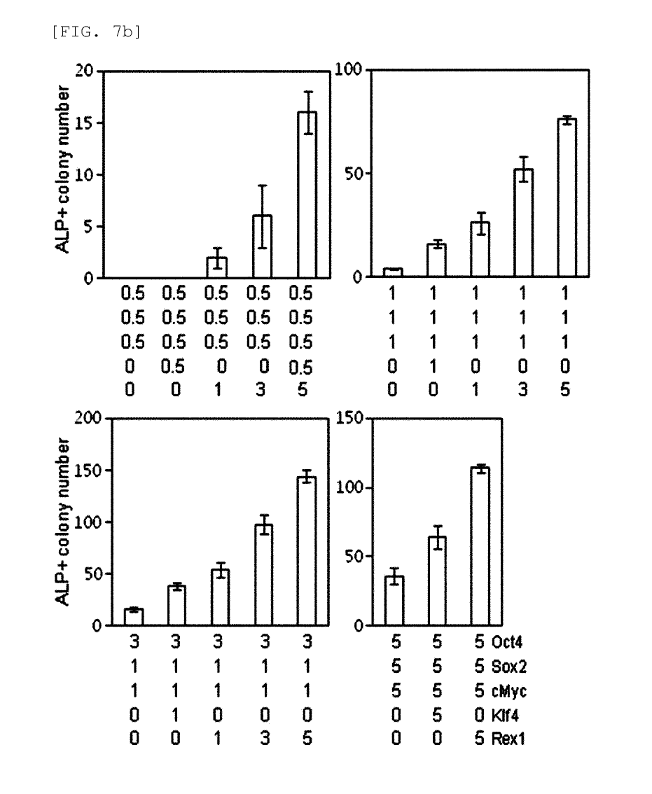

Characteristics of the induced pluripotent stem cell line (pOSM-iPSC) partially reprogrammed by Oct4, Sox2, and cMyc in the absence of Rex1 or Klf4 were analyzed. The results of ALP staining and immunostaining of pOSM-iPSC showed that a partial or no expression of the pluripotency-specific markers including Oct4 and Nanog was observed (FIG. 9a). Further, Real-time RT-PCR was carried out using the primers of Table 1 according to the method of Example 2 to examine mRNA expression. The results showed that total and endogenous expressions of pluripotency marker genes in pOSM-iPSC were not comparable to those in human embryonic stem cells, and retroviral exogenous transgenes were not largely silenced (FIG. 9b). Methylation of the promoter regions of Oct4 and Rex1 genes in pOSM-iPSC were examined by bisulfite sequencing analysis according to the method of Example 8. As shown in FIG. 9c, methylation in pOSM-iPSC cells was found to be maintained. These results indicate that the pOSM-iPSC cell line reprogrammed in the absence of Rex1 or Klf4 was not fully reprogrammed, but partially reprogrammed to acquire the characteristics of human embryonic stem cells.

### Example 16

### Characterization of Induced Pluripotent Stem Cells Established by Rex1

16-1. Marker Expression of Human Embryonic Stem Cells

Pluripotent characteristics of the induced pluripotent stem cell line (OSMK-iPSC; 4F-hiPS) established by Oct4, Sox2, cMyc and Klf4 and the induced pluripotent stem cell line (OSMR-iPSC; 3F+R-hiPS) established by Oct4, Sox2, cMyc and Rex1 were analyzed by ALP staining and immunostaining. Two cell lines that were independently established were subjected to the analysis. The results showed that OSMK-iPSC (4F-hiPS) and OSMR-iPSC (3F+R-hiPS) were very similar to and indistinguishable from each other in the morphology or in the staining of the human embryonic stem cell marker (ALP) and immunostaining (OCT4, NANOG, SSEA-3, SSEA-4, TRA-1-60, TRA-1-81) (FIG. 10). Further, Real-time RT-PCR was carried out using the primers of Table 1 to examine mRNA expressions of Oct4, Sox2, cMyc, Klf4 and Rex1, and the results showed that total and endogenous expression levels of Oct4, Sox2, cMyc, Klf4 and Rex1 in OSMR-iPSC (4F-hiPS) and OSMK-iPSC (3F+R-hiPS) were comparable to those in human embryonic stem cells, and retroviral exogenous transgenes became largely silenced (FIG. 11).

16-2. Genomic Integration of Exogenous Reprogramming Factor in Induced Pluripotent Stem Cells Established by Rex1

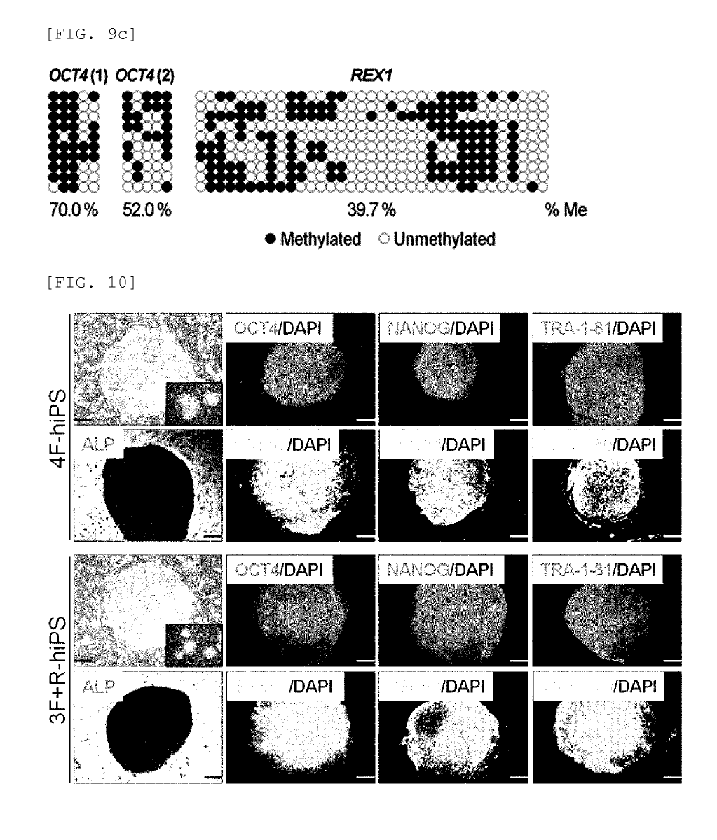

Genomic integrations of exogenous transgenes were examined in two cell lines, OSMK-iPSC (4F-hiPS) and OSMR-iPSC (3F+R-hiPS). Genomic DNAs were extracted using a DNeasy kit (Qiagen, Valencia, Calif.), and each 300 ng of genomic DNAs and transgene-specific primers (Table 2) were used to perform PCR amplification, respectively. The results showed genomic integrations of exogenous Oct4, Sox2, cMyc and Klf4, and exogenous Oct4, Sox2, cMyc and Rex1 in OSMK-iPSC and OSMR-iPSC, respectively (FIG. 12). At this time, human embryonic stem cell line (H9, hES) and human fibroblast cell line (CRL2097, hFF) were used as control groups.

16-3. Normal Karyotype Analysis of Induced Pluripotent Stem Cells Established by Rex1

Karyotype analysis of OSMR-iPSC cell line was performed by the method of Example 9, and the result showed that OSMR-iPSC cell line cultured for 15 passages maintained normal karyotype (FIG. 13).

16-4. Methylation Profiling of Induced Pluripotent Reprogrammed Stem Cells Established by Rex1

According to the method of Example 8, methylation status of the promoter regions of the pluripotency markers, Oct4 and Rex1 genes in OSMK-iPSC and OSMR-iPSC was examined by bisulfite sequencing. As shown in FIG. 14, the results showed that OSMK-iPSC and OSMR-iPSC showed similar patterns to those of human embryonic stem cell line (H9), but the parental hFFs still maintained methylation (FIG. 14).

16-5. Pluripotency of Induced Pluripotent Stem Cells Established by Rex1

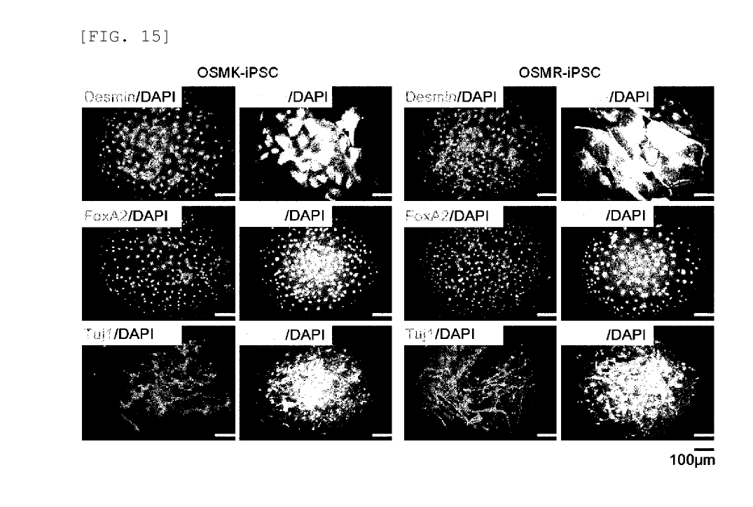

In order to examine whether the OSMK-iPSC and OSMR-iPSC possess the stem cell characteristic of pluripotency, embryonic bodies were prepared from each of the established cell lines, and their additional differentiation capacity was examined. Embryonic bodies were formed in suspension culture, and then adherent-cultured for 10 days using a differentiation medium in a gelatin-coated plate. Immunohistochemical staining and Real-time RT-PCR were performed to examine specific marker expressions in cells differentiated to 3 germ layers. As shown in FIG. 15, the results of immunohistochemical staining showed Tujl (ectoderm), Nestin (ectoderm), desmin (mesoderm), α-SMA (α-smooth muscle actin, mesoderm), Sox17 (endoderm) and FoxA2 (endoderm)-positive cells (FIG. 15). Further, Real-time RT-PCR was carried out using cells (each two cell lines used) further differentiated from embryonic bodies of OSMK-iPSC (4F-hiPS) and OSMR-iPSC (3F+R-hiPS) and the primers of Table 1 to analyze 3 germ layer-specific marker expressions. The results showed expressions of all ectoderm (NCAM, SOX1, TUBB3, OTX1), mesoderm (MSX1, COL1A1, FOXF1, HAND1) and endoderm (AMYLASE, GATA6, SOX17, HGF)-specific genes (FIG. 16), indicating that the human induced pluripotent stem cell line established by Rex1 possesses the pluripotency to differentiate into 3 germ layers. Consequently, these results suggest that Rex1 is functionally significant in the reprogramming process of differentiated somatic cells with no differentiation capacity into induced pluripotent stem cell with the pluripotency to differentiate into 3 germ layers.

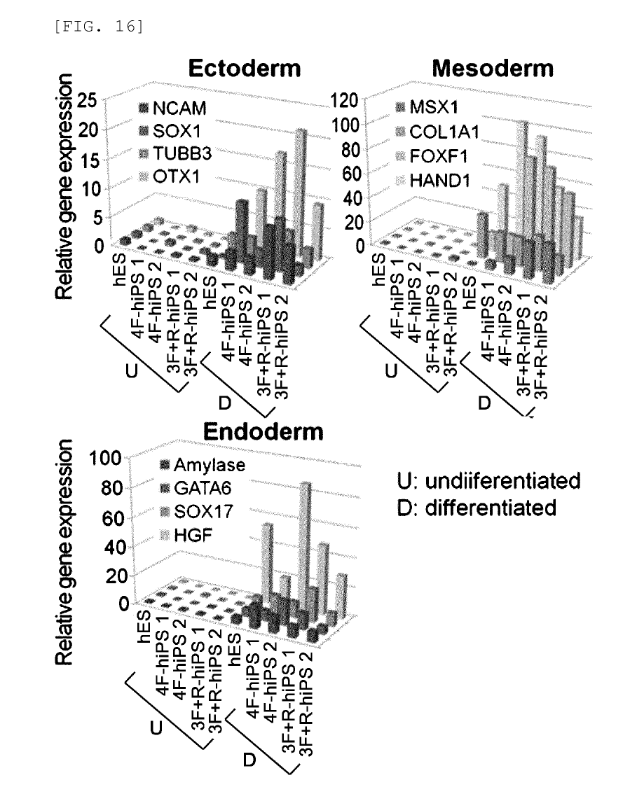

Further, in order to examine in vivo pluripotency of human induced pluripotent stem cell line established by Rex1, OSMR-iPSC was administered into the dorsal flask of immunodeficiency mouse (SCID) via subcutaneous injection. After approximately 12 weeks, teratoma formation was observed, and neural rosette (ectoderm), epidermis containing melanocytes (ectoderm), cartilage (mesoderm) and gut-like epithelium (endoderm) were observed in teratomas by Hematoxylin/eosin staining (FIG. 17), indicating that human induced pluripotent stem cell line reprogrammed by Rex1 possesses the pluripotency to differentiate into 3 germ layers in vivo and in vitro.

### Example 17

### Reprogramming-Improving Effect of Rex1 in Various Differentiated Cells from Different Lineages

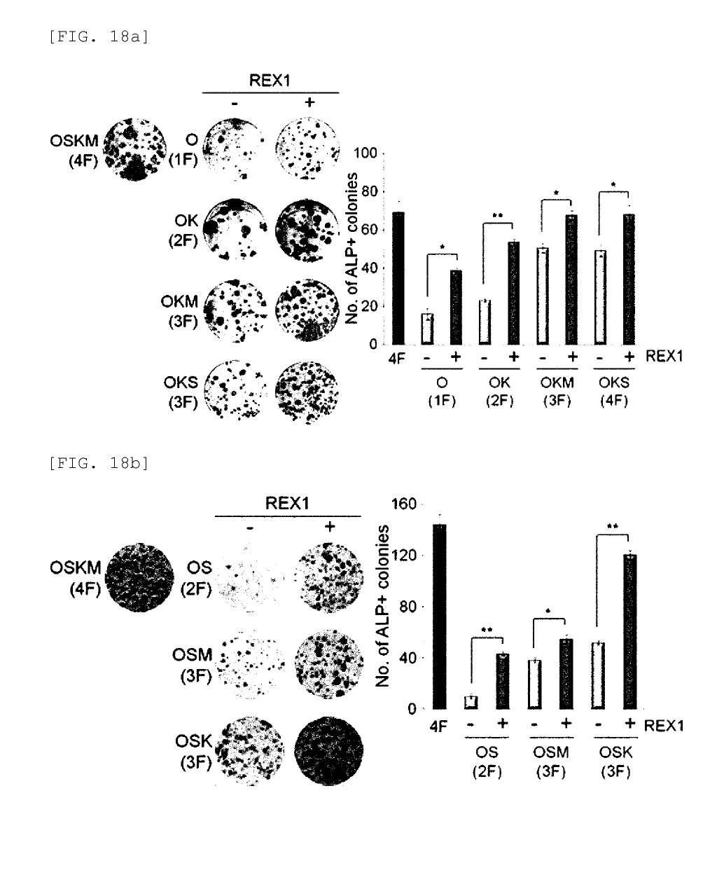

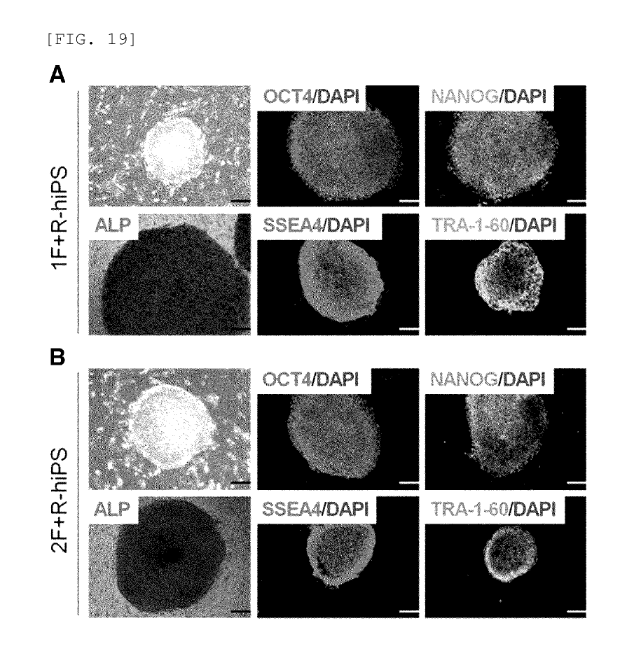

It was examined whether Rex1 shows the reprogramming efficiency-improving effect in human differentiated cell lines differentiated from different lineages. To this end, human neural progenitor cells (ReNcell CX Immortalized cells; hNP) and human mesenchymal stem cells (hMS) were used. As in the previously published results, it is possible to reprogram human neural progenitor cells using only Oct4, but the efficiency is as very low as 0.011% under feeder-free conditions (Nature 461, 649-643, 2009). In the present invention, however, Rex1 increased the number of ALP-positive colonies approximately 2.4-fold under feeder-free conditions. Addition of Rex1 to Oct4 with Klf (OK) and Oct4 and Klf4 with cMyc or Sox2 (OKM or OKS) significantly increased the number of ALP-positive colonies (* p<0.05, * * p<0.01, FIG. 18a). Also, addition of Rex1 to Oct4 and Sox2 in human mesenchymal stem cells significantly increased the number of ALP-positive colonies (** p<0.01). Further, addition of Rex1 to Oct4 and Sox2 with cMyc (OSM) or Oct4 and Sox2 with Klf4 (OSK) significantly increased the number of ALP-positive colonies (* p<0.05, ** p<0.01, FIG. 18b). These results indicate that Rex1 shows the reprogramming efficiency-improving effects in different human differentiated cells such as multipotent stem cells including human neural progenitor cells and human mesenchymal stem cells from different lineages, as well as in human fibroblasts. Further, pluripotency of OR-iPSC (1F+R-hiPS) established from human neural progenitor cells and OSR-iPSC (2F+R-hiPS) established from human mesenchymal stem cells was examined by ALP activity, expression of pluripotency markers such as Oct4, Nanog, SSEA4, and TRA-1-60 (FIG. 19), and demethylation of the promoter regions of Oct4, Nanog, and Rex1 genes (FIG. 20).

### Example 18

### Changes in Cell Growth and Cell Cycle by Specific Suppression of Rex1 Expression

18-1. Suppression of Rex1 Expression by Rex1-Specific shRNA

In order to understand the molecular regulation mechanism of Rex1 associated with maintenance and acquisition of pluripotency, a lentiviral shRNA delivery system was used to specifically suppress the Rex1 expression in pluripotent cells, and then the functions of Rex1 were examined. The human embryonal carcinoma cell line NCCIT (hEC cell) having self-renewal and pluripotency like human embryonic stem cells expresses a large amount of the pluripotency-specific markers, Oct4, Nanog, Sox2 and Rex1. Therefore, NCCIT hEC cells were used in the present invention to investigate suppression of Rex1 expression and associated mechanism.

18-2. Preparation of Lentiviral Rex1-Specific shRNA and Establishment of Rex1 Expression-Suppressed Cell Line by Using the Same

293FT cells were transfected with vectors [TRCN0000107810 (for clone shREX1 #10), TRCN0000107811 (for clone shREX1 #11) and TRCN0000107813 (for clone shREX1 #13), Sigma] having the shRNA base sequence targeting human Rex1 and a vector (shNT, SHC002, Sigma) having non-target control shRNA base sequence as a negative control using lipofectamine 2000, respectively. At this time, lentivirus for each shRNA was prepared using a Mission lentiviral packaging mix (sigma). 24 hours after transfection, the lentivirus-containing medium was collected, and replaced with a new medium. After 24 hours, the second lentivirus-containing medium was collected, and filtered using a 0.45 mm pore-sized filter, and concentrated at 20,000 rpm for 90 minutes, and stored at −70° C. until use. To establish a continuous Rex1 expression-suppressed cell line, NCCIT cells were transfected with lentiviral REX1-targeting shRNA and non-Target shRNA at an MOI of 3 together with polybrene (6 mg/ml). The cell line was selected using the medium containing 1 μg/ml puromycin.

18-3. Suppression of Rex1 Expression by Rex1-Specific shRNA

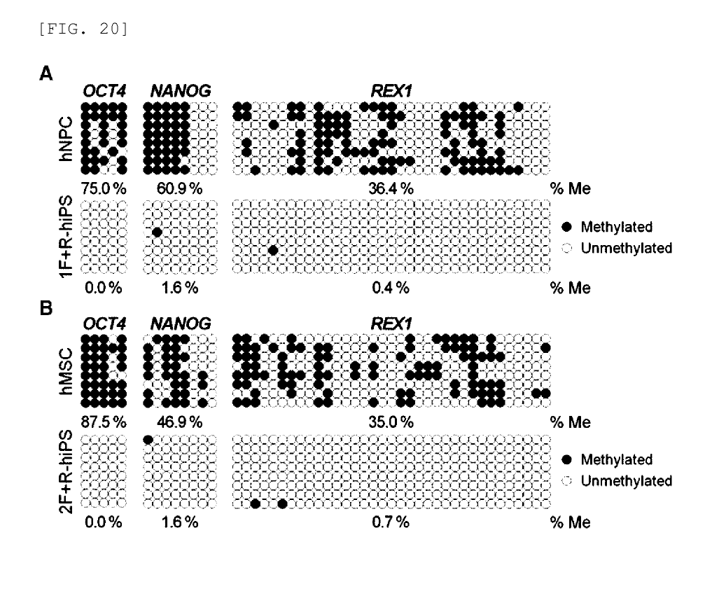

Three shRNA structures (shREX1-10, shREX1-11, and shREX1-13) targeting different regions of Rex1 transcript were used, and each cell line was established by the method of Example 18-2. As a negative control group, a cell line was also established using a non-target control shRNA (shNT) targeting no transcript. The results of Real-time RT-PCR showed that a significant reduction in Rex1 mRNA expression was observed in shREX1-10, shREX1-11, and shREX1-13 cell lines (p<0.01, A of FIG. 21), and the results of Western blot showed that a significant reduction in Rex1 protein expression was also observed in shREX1-10, shREX1-11, and shREX1-13 cell lines (p<0.01, B of FIG. 21).

18-4. Microarray Analysis of Rex1 Expression-Suppressed Cell Line

Total RNAs were extracted from hREX1-10, shREX1-11, and shREX1-13 cell lines and shNT control cell line using a RNA Mini kit (Qiagen), and labeled with Cy3, and hybridized to Agilent Human Whole Genome 4×44K Microarrays (one-color platform) according to the recommendations of the manufacturer. Hybridization images were scanned using an Agilent DNA microarray scanner, and quantified using Feature extraction software (Agilent Technology, Palo Alto, Calif.). All data normalization and selection of genes with fold-changes were performed using GeneSpringGX 7.3 (Agilent Technology, USA). The averages of the normalized ratios were calculated by dividing the average of the normalized signal channel intensity by the average of the normalized control channel intensity. Functional annotation of genes was performed according to Gene Ontology™ Consortium (http://www.geneontology.org/index.shtml) by GeneSpringGX 7.3. Gene classification based on searches done by BioCarta (http://www.biocarta.com/), GenMAPP (http://www.genmapp.org/), DAVID (http://david.abcc.ncifcrf.gov/) and Medline databases (http://www.ncbi.nlm.nih.gov/).

18-5. Cell Differentiation by Suppression of Rex1 Expression

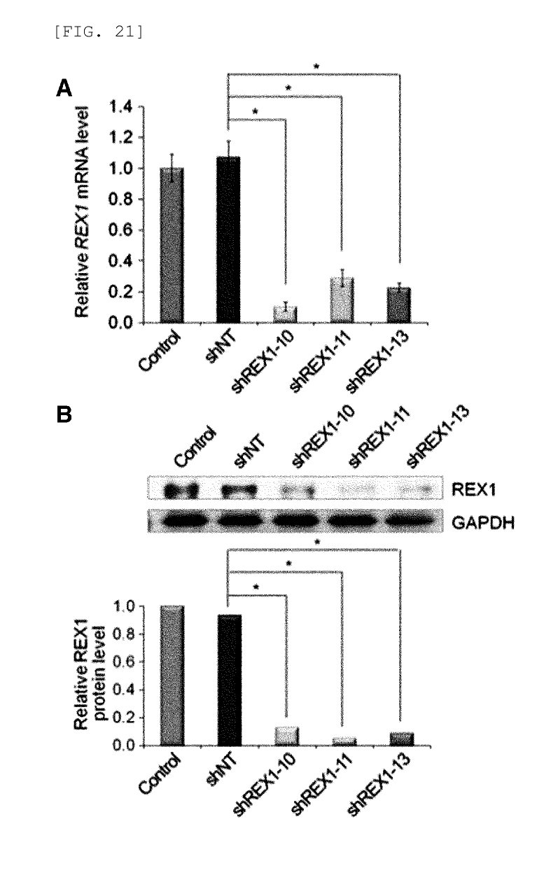

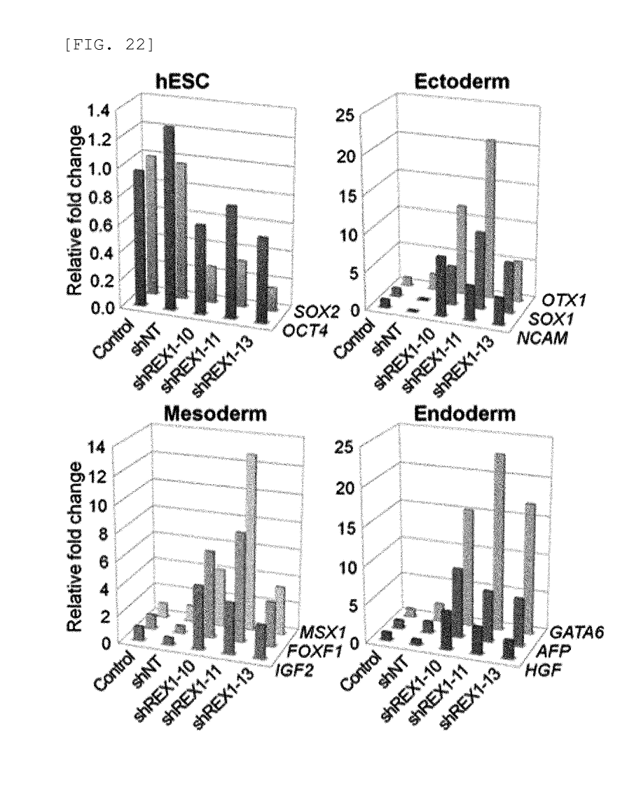

The results of Real-time RT-PCR showed that average 1.5-fold and 4-fold reduction in the expression of pluripotency markers, Oct4 and Sox2 were observed in the Rex1 expression-suppressed cell lines, and the results of 3 germ layer marker expression showed that the expressions of ectodermal markers (NCAM, SOX1, OTX1), mesodermal markers (IGF2, FOXF1, MSX1), endodermal markers (HGF, AFP, GATA6) increased from 2.4-fold to 22.9-fold, compared to the control group (FIG. 22). Microarray-based global gene expression profiling was performed using these Rex1 expression-suppressed cell lines by the method of Example 18-4. As in the results of FIG. 22, the result of heat map analysis of gene expression by microarray showed a reduction in the expression of pluripotency markers, and an increase in the expression of 3 germ layer-specific differentiation marker genes (FIG. 23).

18-6. Changes in Expression of Cell Cycle-Related Genes by Suppression of Rex1 Expression

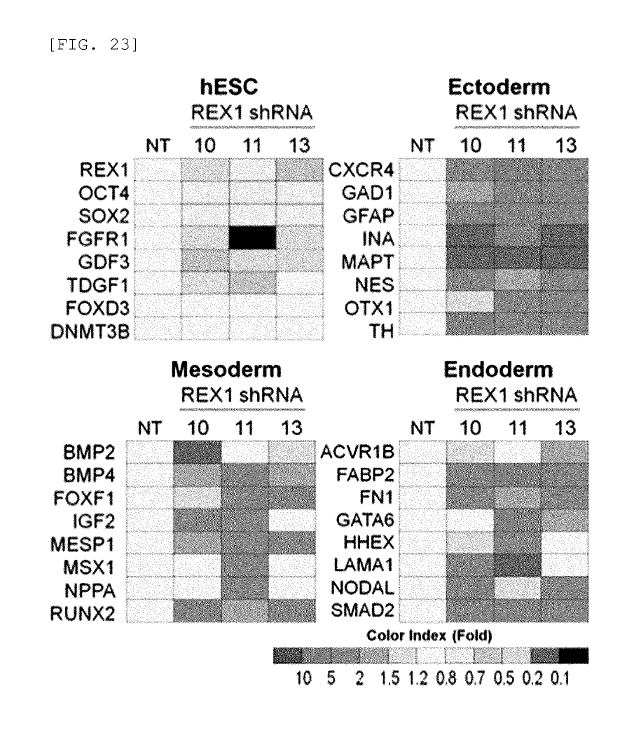

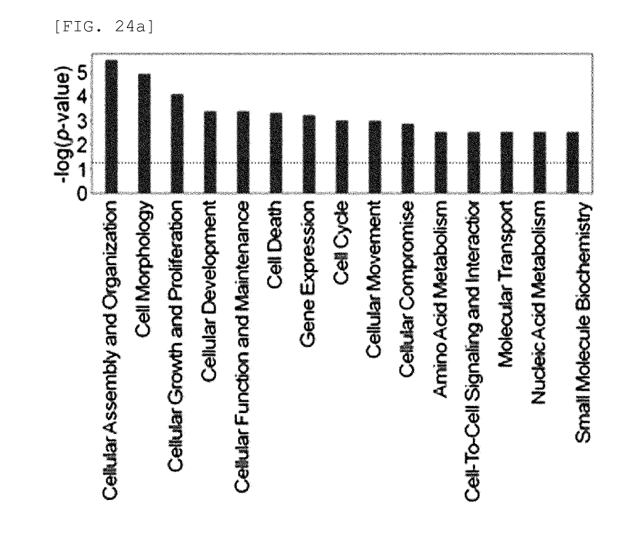

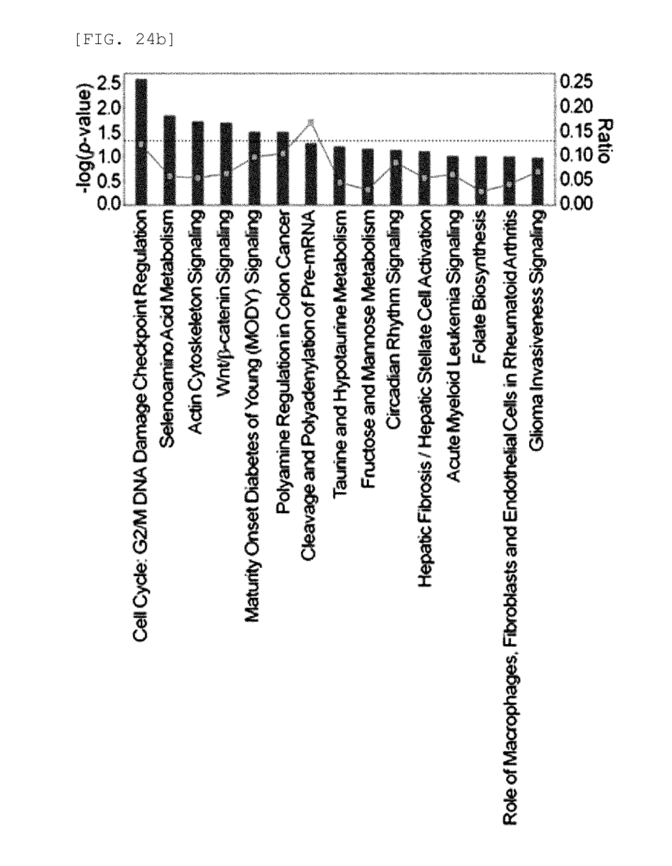

Functional classification of the genes showing different expressions in the Rex1 expression-suppressed cell lines (hREX1-10, shREX1-11, and shREX1-13) was performed based on “Ingenuity Pathway Analysis software”. In terms of biological functional category, genes related to cellular assembly and organization, cell morphology, cellular growth and proliferation, cellular development and cellular function and maintenance showed significant changes (FIG. 24a), and in terms of canonical pathways, cell cycle, in particular, G2/M DNA damage checkpoint regulation showed the most significant changes (FIG. 24b). In particular, the results of heat map analysis by microarray also showed that the gene expression patterns related to the top canonical pathways, G2/M cell cycle were changed by suppression of Rex1 expression, as shown in FIG. 25. In particular, expressions of CCNB1 (cyclin B1) and CCNB2 (cyclin B2) that participate in the regulation of G2/M progression were inhibited by suppression of Rex1 expression. In addition, expressions of YWHAH (14-3-3 eta), BTRC (beta-transducin repeat containing), and TOP2B (topoisomerase II beta) that are positive regulators of G2/M progression were inhibited. On the contrary, expressions of MDM4, of which overexpression inhibits MDM2-mediated p53 degradation to delay cell cycle, and PCAF, of which overexpression arrests cell cycle progression, were increased by suppression of Rex1 expression (FIG. 25). All these results suggest that cell cycle can be inhibited by suppression of Rex1 expression.

18-7. Cell Growth Inhibition by Suppression of Rex1 Expression

In order to examine changes in cell growth by suppression of Rex1 expression, an equal number of cells (50,000 cells/12 well) was plated, and the number of cells after 1, 3, 5 days was determined using a trypan blue solution. The result showed that cell growth was significantly reduced in Rex1 expression-suppressed cell lines (hREX1-10, shREX1-11, and shREX1-13) (** P<0.01, *P<0.05) (FIG. 26).

18-8. Changes in Cell Cycle Distribution by Suppression of Rex1 Expression

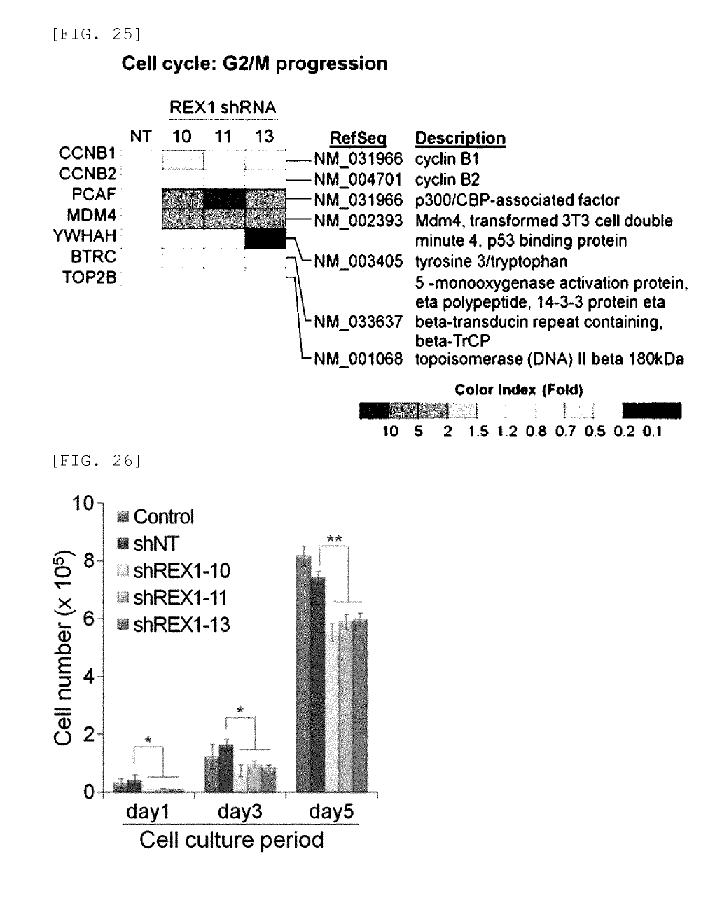

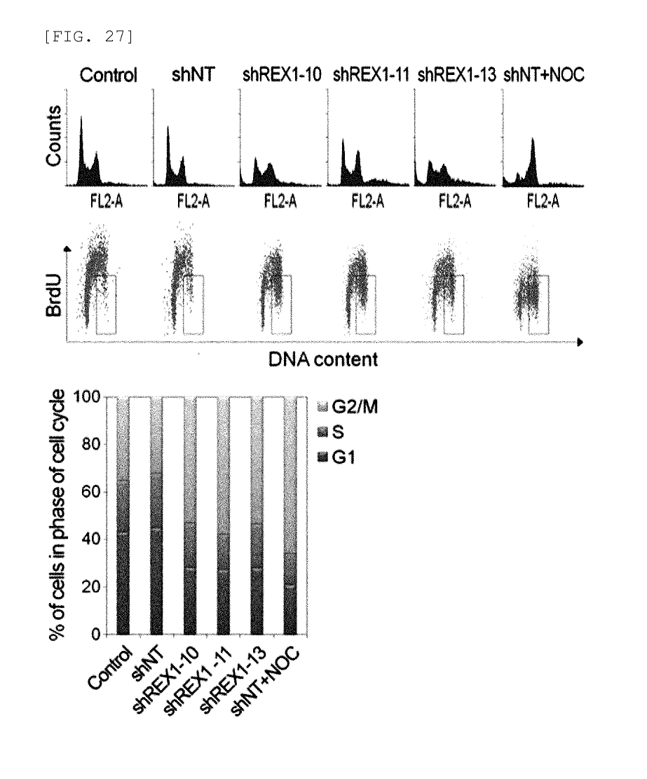

For analysis of cell cycle distribution, propidium iodide (PI) and a nucleoside analogue bromodeoxyuridine (BrdU) monoclonal antibody were used. First, for measurement of the DNA amount by PI, cells were suspended at a density of 1×10⁵ cell/mL, and washed with PBS (1200 rpm, 5 min), and then fixed in 1 mL of 70% alcohol at 4° C. overnight. Next day, 3 mL of PBS was further added, followed by centrifugation. Then, 50 μL of PI (400 mg/mL), 50 μL of RNase A (1 mg/mL), and 1 μL of Triton X-100 (10%) were added, and reacted at room temperature for 30 minutes. The amount of DNA in the stained sample was determined by flow cytometry. During the S Phase, cellular metabolism is focused on DNA replication, and thus the relationship between S phase and DNA replication can be examined by analysis of BrdU incorporation. For flow cytometry to determine cell proliferation during DNA replication in the S phase, it was intended to examine incorporation of a thymidine analogue, BrdU into DNA. The cultured cells were treated with 10 mM BrdU for 4 hours, and then cells were harvested and fixed with 3% formaldehyde diluted in PBS at 4° C. for 1 hour. Thereafter, the cells were centrifuged, and treated with 1% Triton X-100 at room temperature for 5 minutes. The cells were centrifuged again, and harvested and washed with PBS, and treated with 4N HCl for 10 minutes at room temperature to denature double-stranded DNAs, followed by neutralization with sodium tetraborate. The cells were treated with a blocking solution (4% BSA, 0.01% Tween 20 in PBS) at room temperature for 30 minutes, and reacted with anti-BrdU mouse IgG for 30 minutes at 4° C., and then reacted with Fluorescein (FITC)-conjugated goat anti-mouse IgG for 30 minutes at 4° C., followed by flow cytometry. As shown in FIG. 27, the results of analyzing cell cycle distribution by flow cytometry using PI and BrdU showed that the G1 phase was reduced, the G2/M phase was significantly increased, and the S phase was slightly reduced in the Rex1 expression-suppressed cell lines (hREX1-10, shREX1-11, and shREX1-13) (P<0.01, FIG. 27).

18-9. G2 Phase Arrest by Suppression of Rex1 Expression

In order to examine whether suppression of Rex1 expression causes cell cycle arrest in the G2 phase or M (mitosis) phase, phosphorylation of histone H3 at serine 10 was analyzed. The phosphorylation of histone H3 at serine 10 (ser10) is a marker for chromosome condensation which occurs during the M phase (Chromosoma 1997; 106:348-360). Immunostaining and Western blotting were performed using antibodies capable of detecting histone H3 (ser10) phosphorylation. The results of immunostaining showed that approximately 11% of the control group (control and shNT cell groups) and approximately 37% of the cells treated with nocodazole for M phase arrest were histone H3 (ser10) phosphorylation-positive cells. On the contrary, less than 4% of the Rex1 expression-suppressed cell lines (hREX1-10, shREX1-11, and shREX1-13) were histone H3 (ser10) phosphorylation-positive cells (A of FIG. 28). These results are consistent with those of Western blotting using histone H3 (ser10) phosphorylation-specific antibodies (B of FIG. 28). Taken together, G2 phase arrest is caused by suppression of Rex1 expression.

18-10. Changes in Expression of G2/M Progression-Related Protein by Suppression of Rex1 Expression

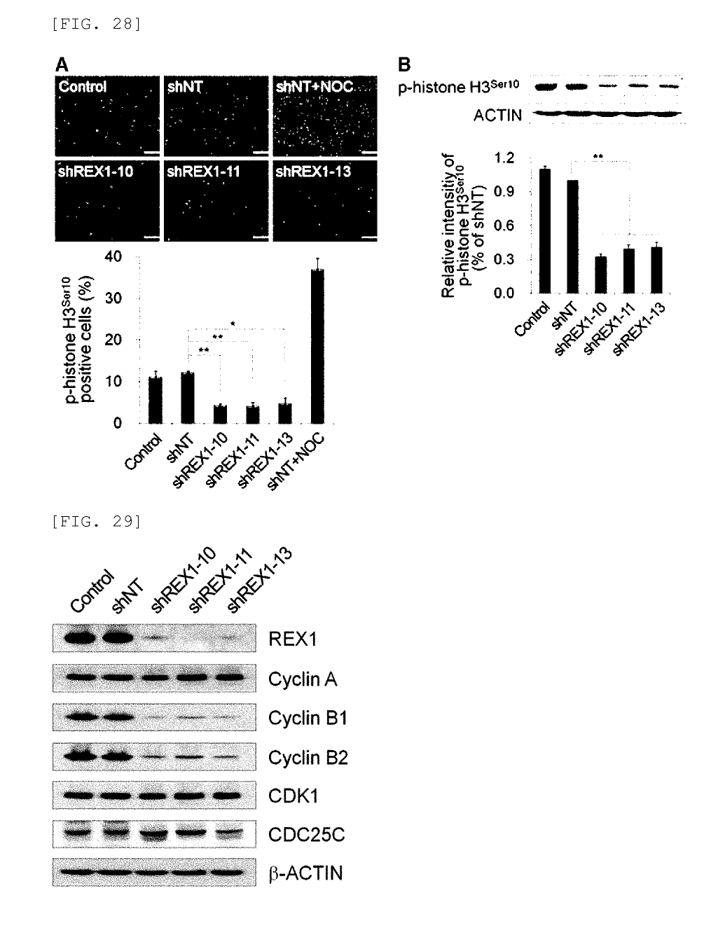

Changes in expression of G2/M progression-related protein by suppression of Rex1 expression were examined by Western blotting. Cyclin B, a key regulator for cell cycle progression at G2/M phase, has two isoforms of cyclin B1 and cyclin B2. Expression levels of these two proteins were remarkably reduced in the Rex1 expression-suppressed cell lines (hREX1-10, shREX1-11, and shREX1-13). However, no changes in the expression levels of other key regulators for cell cycle progression at G2/M phase, CDK1, CDC25C and cyclin A were observed in the Rex1 expression-suppressed cell lines (FIG. 29). Activities of cyclin B1 and cyclin B2 are mainly regulated by their expression levels, and thus these results suggest that expressions of the key regulators for cell cycle progression at G2/M phase, cyclin B1 and cyclin B2 are regulated by Rex1, and cell proliferation is maintained by the promoted cell cycle progression at G2/M phase.

### Example 19

### Induction of Cyclin B1 and Cyclin B2 Expressions by Rex1

19-1. Induction of Cyclin B1 and Cyclin B2 Expressions by Rex1 in Human Fibroblast

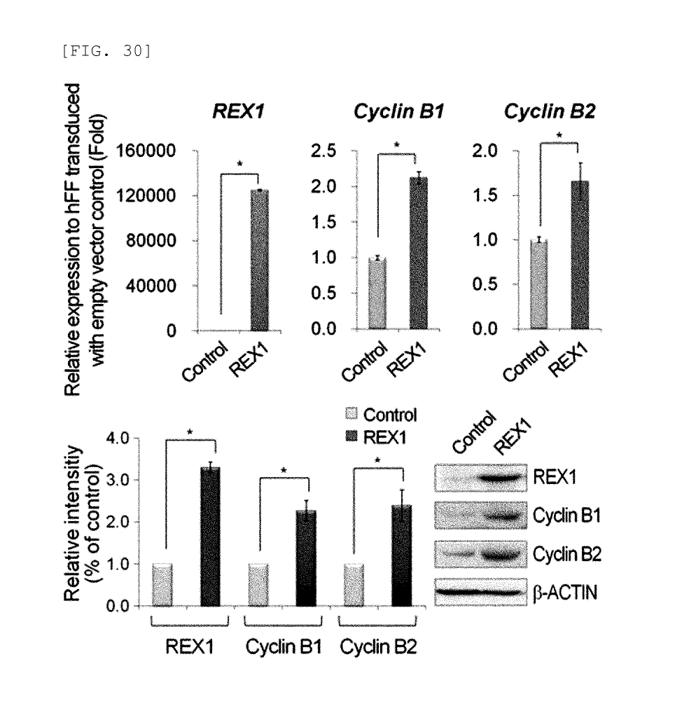

In order to examine induction of cyclin B1 and cyclin B2 expressions by Rex1, a retroviral vector encoding Rex1 was transfected into human fibroblasts. 5 days after transfection, the expression level of Rex1 transcript was examined by real-time RT-PCR, and the result showed Rex1 overexpression (top of FIG. 30). 5 days after transfection, the increased expression of Rex1 protein was also confirmed by Western blotting (bottom of FIG. 30). The mRNA levels of cyclin B1 and cyclin B2 were significantly increased to 2.1-fold and 1.7-fold by Rex1 overexpression, respectively (p<0.01, top of FIG. 30), and the cyclin B1 and cyclin B2 proteins were significantly increased to 2.3-fold and 2.4-fold, respectively (p<0.01, bottom of FIG. 30).

19-2. Increased Expressions of Cyclin B1 and Cyclin B2 by Rex1 During Reprogramming of Human Fibroblasts

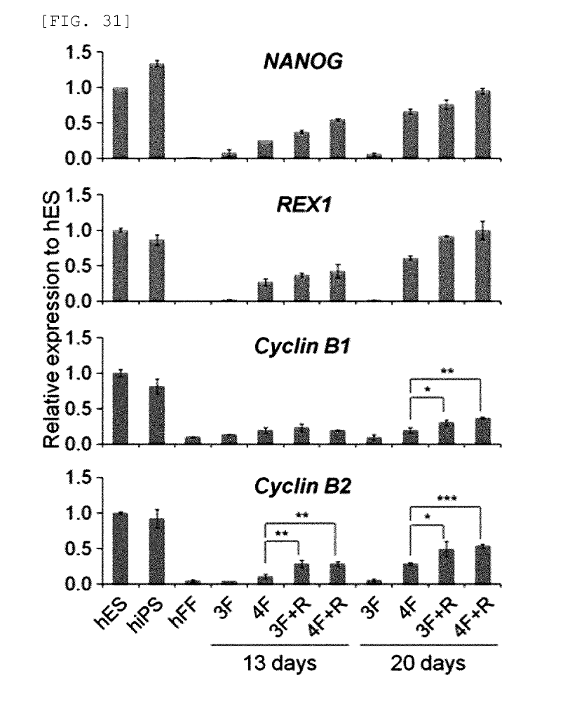

In order to examine whether the use of Rex1 as a reprogramming factor is able to induce more greatly or more rapidly the cyclin B1 and cyclin B2 expressions during reprogramming process, reprogramming of human fibroblasts was carried out using Rex1 and various combinations of other reprogramming factors (Oct4, Sox2, cMyc, Klf4). 13 days and 20 days after transduction with reprogramming factors of Oct4, Sox2, cMyc (OSM, 3F), Oct4, Sox2, cMyc, Klf4 (OSMK, 4F), Oct4, Sox2, cMyc, Rex1 (OSMR, 3F+R), and Oct4, Sox2, cMyc, Klf4, Rex1 (OSMKR, 4F+R), total RNAs were extracted from each fibroblast, and expression levels of pluripotency markers, cyclin B1 and cyclin B2 were examined by real-time RT-PCR. The results showed that expressions of the stem cell marker Nanog and Rex1 were induced in all transductions, excluding 3F transduction, and in particular, a significant increase in the expression levels of cyclin B1 and cyclin B2 was observed in 3F+R transduction (transduction of Rex1 instead of Klf4) and in 4F+R transduction (transduction of Rex1 in addition to 4F), compared to 4F transduction (* p<0.1, ** p<0.05, *** p<0.01, FIG. 31). These results suggest that G2/M progression is accelerated by increased expressions of the key M phase regulators, cyclin B1 and cyclin B2 by Rex1, leading to an improvement in cell proliferation. Further, Rex1 improves cell-cycle arrest and cell proliferation inhibition which occur in the previous reprogramming technology, and thus it is expected that Rex1 contributes to an improvement in reprogramming efficiency.

### Effect of the Invention

According to the present invention, Rex1 can be effectively used for the purpose of improving the previous reprogramming technology, because the use of Rex1 markedly increases the number of induced pluripotent stem cells fully reprogrammed from differentiated cells and, compared to reprogramming in the absence of Rex1, reduces the time required for the reprogramming, and replaces the previous reprogramming factors such as the oncogene Klf or reduces the number of factors required for reprogramming. Further, the present invention demonstrated that Rex1 can be used as a universal reprogramming factor effectively acting on production of induced pluripotent stem cells from a small amount of differentiated cells obtained from various cell lineages. Accordingly, there is an advantage in that difficult or impossible reprogramming of somatic cells by the previous factors can be improved by use of Rex1. In conclusion, the present invention provides Rex1 as a universal reprogramming factor, and thus contributes to advances in reprogramming technology capable of effectively producing clinically safe induced pluripotent stem cells from patients' somatic cells. It is possible to apply the induced pluripotent stem cells derived therefrom and differentiated cells in the development of personalized stem cell therapy products and new drugs, and therefore contributes to advances in practical applications of the related products.

1. A method for inducing reprogramming differentiated cells into induced pluripotent stem cells, comprising introducing a reprogramming-inducing factor comprising the Rex1 (reduced expression protein 1) protein or a nucleic acid molecule encoding the Rex1 protein into differentiated cells.

2. The method according to claim 1, wherein the nucleic acid molecule encoding the Rex1 protein is included in an expression vector.

3. The method according to claim 1, wherein the nucleic acid molecule encoding the Rex1 protein is messenger RNA (mRNA).

4. The method according to claim 1, wherein the reprogramming-inducing factor further comprises one or more proteins selected from the group consisting of Oct4, Sox2, KlF4, c-Myc, Nanog, and Lin-28, or one or more nucleic acid molecules encoding said proteins.

5. The method according to claim 1, wherein the cells are derived from human.

6. The method according to claim 1, wherein the differentiated cells are somatic cells or somatic stem cells.

7. The method according to claim 6, wherein the somatic stem cells are neural stem cells or mesenchymal stem cells.

8. A method for producing induced pluripotent stem cells that are reprogrammed from differentiated cells, comprising:
(a) inducing reprogramming differentiated cells into induced pluripotent stem cells by the method according to claim 1, and
(b) culturing the cells of step (a).

9. The method according to claim 8, further comprising (c) isolating embryonic stem cell-like colonies from the culture broth obtained in step (b).

10. The method according to claim 8, wherein the reprogramming-inducing factor further comprises one or more proteins selected from the group consisting of Oct4, Sox2, KlF4, c-Myc, Nanog, and Lin-28, or one or more nucleic acid molecules encoding said proteins.

11. The method according to claim 8, wherein the reprogramming-inducing factor is added to the culture broth of differentiated cells in step (a).

12. The method according to claim 8, wherein the reprogramming-inducing factor is directly injected into the differentiated cells in step (a).

13. The method according to claim 8, wherein the differentiated cells are transfected with a virus obtained from a packaging cell transfected with a viral vector having a gene encoding the reprogramming-inducing factor in step (a).

14. The method according to claim 8, wherein the nucleic acid molecule encoding the Rex1 protein is messenger RNA (mRNA).

15. The method according to claim 8, wherein the cells are derived from human.

16. The method according to claim 8, wherein the differentiated cells are somatic cells or somatic stem cells.

17. The method according to claim 16, wherein the somatic stem cells are neural stem cells or mesenchymal stem cells.

18. (canceled)

19. The method according to claim 1, wherein the reprogramming-inducing factor is added to the culture broth of differentiated cells.

20. The method according to claim 1, wherein the reprogramming-inducing factor is directly injected into the differentiated cells.

21. The method according to claim 1, wherein the differentiated cells are transfected with a virus obtained from a packaging cell transfected with a viral vector having a gene encoding the reprogramming-inducing factor.


SEQUENCE LISTING (77 sequences)

SEQ ID NO: 1 - DNA, 933 bp, Homo sapiens
  gene (1)..(933): Rex1, Homo sapiens ZFP42 zinc finger protein (ZFP42),  transcript variant X3, mRNA, ACCESSION   XM_005262748, VERSION XM_005262748.1  GI530377186
atgagccagcaactgaagaaacgggcaaagacaagacaccagaaaggcctgggtggaagagcccccagtggggctaagcccaggcaaggcaagtcaagccaagacctgcaggcggaaatagaacctgtcagcgcggtgtgggccttatgtgatggctatgtgtgctatgagcctggccctcaggctctcggaggggatgatttctcagactgttacatagaatgcgtcataaggggtgagttttctcaacccatcctggaagaggactcactttttgagtccttggaatacctaaagaaaggatcagaacaacagctttctcaaaaggttttcgaagcaagctcccttgaatgttctttggaatacatgaaaaaaggggtaaagaaagagcttccacaaaagatagttggagagaattcgcttgagtattctgagtacatgacaggcaagaagcttccgcctggaggaatacctggcattgacctatcagatcctaaacagctcgcagaatttgctagaaagaagccccccataaataaagaatatgacagtctgagcgcaatcgcttgtcctcagagtggatgcactaggaagttgaggaatagagctgccctgagaaagcatctcctcattcatggtccccgagaccacgtctgtgcggaatgtgggaaagcgttcgttgagagctcaaaactaaagagacatttcctggttcatactggagagaagccgtttcggtgcacttttgaagggtgcggaaagcgcttctctctggactttaatttgcgtacgcacgtgcgcatccacacgggggagaaacgtttcgtgtgtccctttcaaggctgcaacaggaggtttattcagtcaaataacctgaaagcccacatcctaacgcatgcaaatacgaacaagaatgaacaagagggaaagtag

SEQ ID NO: 2 - DNA, 36 bp, Artificial Sequence
  Forward primer for amplification of Rex1 cDNA
aattggatccatgagccagcaactgaagaaacgggc

SEQ ID NO: 3 - DNA, 41 bp, Artificial Sequence
  Reverse primer for amplification of Rex1 cDNA
aattgcggccgcctactttccctcttgttcattcttgttcg

SEQ ID NO: 4 - DNA, 25 bp, Artificial Sequence
  Forward primer for amplification of bi Oct4-3
atttgttttttgggtagttaaaggt

SEQ ID NO: 5 - DNA, 27 bp, Artificial Sequence
  Reverse primer for amplification of bi Oct4-3
ccaactatcttcatcttaataacatcc

SEQ ID NO: 6 - DNA, 27 bp, Artificial Sequence
  Forward primer for amplification of bi Oct4-4
ggatgttattaagatgaagatagttgg

SEQ ID NO: 7 - DNA, 25 bp, Artificial Sequence
  Reverse primer for amplification of bi Oct4-4
cctaaactccccttcaaaatctatt

SEQ ID NO: 8 - DNA, 25 bp, Artificial Sequence
  Forward primer for amplification of Nanog
tggttaggttggttttaaatttttg

SEQ ID NO: 9 - DNA, 26 bp, Artificial Sequence
  Reverse primer for amplification of Nanog
aacccacccttataaattctcaatta

SEQ ID NO: 10 - DNA, 23 bp, Artificial Sequence
  forward primer for total OCT4
gagaaggatgtggtccgagtgtg

SEQ ID NO: 11 - DNA, 22 bp, Artificial Sequence
  reverse primer for total OCT4
cagaggaaaggacactggtccc

SEQ ID NO: 12 - DNA, 20 bp, Artificial Sequence
  forward primer for total SOX2
agaaccccaagatgcacaac

SEQ ID NO: 13 - DNA, 20 bp, Artificial Sequence
  reverse primer for total SOX2
atgtaggtctgcgagctggt

SEQ ID NO: 14 - DNA, 20 bp, Artificial Sequence
  forward primer for total KLF4
accctgggtcttgaggaagt

SEQ ID NO: 15 - DNA, 20 bp, Artificial Sequence
  reverse primer for total KLF4
acgatcgtcttcccctcttt

SEQ ID NO: 16 - DNA, 20 bp, Artificial Sequence
  forward primer for total cMYC
cctaccctctcaacgacagc

SEQ ID NO: 17 - DNA, 20 bp, Artificial Sequence
  reverse primer for total cMYC
ctctgaccttttgccaggag

SEQ ID NO: 18 - DNA, 20 bp, Artificial Sequence
  forward primer for total REX1
aatgcgtcataaggggtgag

SEQ ID NO: 19 - DNA, 20 bp, Artificial Sequence
  reverse primer for total REX1
tcaatgccaggtattcctcc

SEQ ID NO: 20 - DNA, 24 bp, Artificial Sequence
  forward primer for endo OCT4
gacagggggaggggaggagctagg

SEQ ID NO: 21 - DNA, 26 bp, Artificial Sequence
  reverse primer for endo OCT4
cttccctccaaccagttgccccaaac

SEQ ID NO: 22 - DNA, 26 bp, Artificial Sequence
  forward primer for endo SOX2
gggaaatgggaggggtgcaaaagagg

SEQ ID NO: 23 - DNA, 26 bp, Artificial Sequence
  reverse primer for endo SOX2
ttgcgtgagtgtggatgggattggtg

SEQ ID NO: 24 - DNA, 22 bp, Artificial Sequence
  forward primer for endo KLF4
agcctaaatgatggtgcttggt

SEQ ID NO: 25 - DNA, 23 bp, Artificial Sequence
  reverse primer for endo KLF4
ttgaaaactttggcttccttgtt

SEQ ID NO: 26 - DNA, 15 bp, Artificial Sequence
  forward primer for endo cMYC
cgggcgggcactttg

SEQ ID NO: 27 - DNA, 19 bp, Artificial Sequence
  reverse primer for endo cMYC
ggagagtcgcgtccttgct

SEQ ID NO: 28 - DNA, 20 bp, Artificial Sequence
  forward primer for endo REX1
tcagtgacaaacatgcctca

SEQ ID NO: 29 - DNA, 20 bp, Artificial Sequence
  reverse primer for endo REX1
ccttcctgttggaagcacac

SEQ ID NO: 30 - DNA, 23 bp, Artificial Sequence
  forward primer for trans OCT4
gagaaggatgtggtccgagtgtg

SEQ ID NO: 31 - DNA, 24 bp, Artificial Sequence
  reverse primer for trans OCT4
ccctttttctggagactaaataaa

SEQ ID NO: 32 - DNA, 26 bp, Artificial Sequence
  forward primer for trans SOX2
ggcacccctggcatggctcttggctc

SEQ ID NO: 33 - DNA, 24 bp, Artificial Sequence
  reverse primer for trans SOX2
ttatcgtcgaccactgtgctgctg

SEQ ID NO: 34 - DNA, 25 bp, Artificial Sequence
  forward primer for trans KLF4
acgatcgtggccccggaaaaggacc

SEQ ID NO: 35 - DNA, 24 bp, Artificial Sequence
  reverse primer for trans KLF4
ttatcgtcgaccactgtgctgctg

SEQ ID NO: 36 - DNA, 27 bp, Artificial Sequence
  forward primer for trans cMYC
caacaaccgaaaatgcaccagccccag

SEQ ID NO: 37 - DNA, 24 bp, Artificial Sequence
  reverse primer for trans cMYC
ttatcgtcgaccactgtgctgctg

SEQ ID NO: 38 - DNA, 20 bp, Artificial Sequence
  forward primer for trans REX1
acgtttcgtgtgtccctttc

SEQ ID NO: 39 - DNA, 20 bp, Artificial Sequence
  reverse primer for trans REX1
taacctgaaagcccacatcc

SEQ ID NO: 40 - DNA, 20 bp, Artificial Sequence
  forward primer for NANOG
caaaggcaaacaacccactt

SEQ ID NO: 41 - DNA, 20 bp, Artificial Sequence
  reverse primer for NANOG
attgttccaggtctggttgc

SEQ ID NO: 42 - DNA, 20 bp, Artificial Sequence
  forward primer for DNMT3B
ataagtcgaaggtgcgtcgt

SEQ ID NO: 43 - DNA, 20 bp, Artificial Sequence
  reverse primer for DNMT3B
ggcaacatctgaagccattt

SEQ ID NO: 44 - DNA, 20 bp, Artificial Sequence
  forward primer for NCAM
aggagacagaaacgaagcca

SEQ ID NO: 45 - DNA, 20 bp, Artificial Sequence
  reverse primer for NCAM
ggtgttggaaatgctctggt

SEQ ID NO: 46 - DNA, 20 bp, Artificial Sequence
  forward primer for SOX1
gggaaaacgggcaaaataat

SEQ ID NO: 47 - DNA, 20 bp, Artificial Sequence
  reverse primer for SOX1
ccatctgggcttcaagtgtt

SEQ ID NO: 48 - DNA, 24 bp, Artificial Sequence
  forward primer for OTX1
agacgcatcagaccctgaaggact

SEQ ID NO: 49 - DNA, 21 bp, Artificial Sequence
  reverse primer for OTX1
ccagacctggactctagactc

SEQ ID NO: 50 - DNA, 20 bp, Artificial Sequence
  forward primer for TUBB3
ggccaagttctgggaagtca

SEQ ID NO: 51 - DNA, 19 bp, Artificial Sequence
  reverse primer for TUBB3
cgagtcgcccacgtagttg

SEQ ID NO: 52 - DNA, 20 bp, Artificial Sequence
  forward primer for MSX1
tcctcaagctgccagaagat

SEQ ID NO: 53 - DNA, 20 bp, Artificial Sequence
  tactgcttct ggcggaactt
tactgcttctggcggaactt

SEQ ID NO: 54 - DNA, 20 bp, Artificial Sequence
  forward primer for IGF2
cagacccccaaattatcgtg

SEQ ID NO: 55 - DNA, 20 bp, Artificial Sequence
  reverse primer for IGF2
gccaagaaggtgagaagcac

SEQ ID NO: 56 - DNA, 20 bp, Artificial Sequence
  forward primer for COL1A1
ggacacaatggattgcaagg

SEQ ID NO: 57 - DNA, 20 bp, Artificial Sequence
  reverse primer for COL1A1
taaccactgctccactctgg

SEQ ID NO: 58 - DNA, 20 bp, Artificial Sequence
  forward primer for FOXF1
aaaggagccacgaagcaagc

SEQ ID NO: 59 - DNA, 21 bp, Artificial Sequence
  reverse primer for FOXF1
aggctgaagcgaaggaagagg

SEQ ID NO: 60 - DNA, 20 bp, Artificial Sequence
  forward primer for HAND1
tcccttttccgcttgctctc

SEQ ID NO: 61 - DNA, 20 bp, Artificial Sequence
  reverse primer for HAND1
catcgcctacctgatggacg

SEQ ID NO: 62 - DNA, 20 bp, Artificial Sequence
  forward primer for HGF
gcatcaaatgtcagccctgg

SEQ ID NO: 63 - DNA, 20 bp, Artificial Sequence
  reverse primer for HGF
caacgctgacatggaattcc

SEQ ID NO: 64 - DNA, 20 bp, Artificial Sequence
  forward primer for GATA6
ccatgactccaacttccacc

SEQ ID NO: 65 - DNA, 20 bp, Artificial Sequence
  reverse primer for GATA6
acggaggacgtgacttcggc

SEQ ID NO: 66 - DNA, 23 bp, Artificial Sequence
  forward primer for AMYLASE
gctgggctcagtattccccaaat

SEQ ID NO: 67 - DNA, 24 bp, Artificial Sequence
  reverse primer for AMYLASE
gacgacaatctctgacctgagtag

SEQ ID NO: 68 - DNA, 20 bp, Artificial Sequence
  forward primer for AFP
agcagcttggtggtggatga

SEQ ID NO: 69 - DNA, 21 bp, Artificial Sequence
  reverse primer for AFP
cctgagcttggcacagatcct

SEQ ID NO: 70 - DNA, 21 bp, Artificial Sequence
  forward primer for SOX17
ttcgtgtgcaagcctgagatg

SEQ ID NO: 71 - DNA, 19 bp, Artificial Sequence
  reverse primer for SOX17
gtcggacaccaccgaggaa

SEQ ID NO: 72 - DNA, 20 bp, Artificial Sequence
  forward primer for cyclin B1
tggcgctccgagtcaccagg

SEQ ID NO: 73 - DNA, 20 bp, Artificial Sequence
  reverse primer for cyclin B1
gttgcagcaggggccgtagg

SEQ ID NO: 74 - DNA, 21 bp, Artificial Sequence
  forward primer for cyclin B2
aggcgagcatcaaaagccggg

SEQ ID NO: 75 - DNA, 22 bp, Artificial Sequence
  reverse primer for cyclin B2
gggaggcaaggtctttgacggc

SEQ ID NO: 76 - DNA, 19 bp, Artificial Sequence
  forward primer for GAPDH
gaaggtgaaggtcggagtc

SEQ ID NO: 77 - DNA, 20 bp, Artificial Sequence
  reverse primer for GAPDH
gaagatggtgatgggatttc